(19) United States
(12) Patent Application Publication
(10) Pub. No.: US 2021/0121059 A1
(43) Pub. Date: Apr. 29, 2021

(54) OPHTHALMIC INSTRUMENT, MANAGEMENT DEVICE, AND METHOD FOR MANAGING AN OPHTHALMIC INSTRUMENT

(71) Applicant: NIKON CORPORATION, Minato-ku, Tokyo (JP)

(72) Inventors: Shota MIYAZAKI, Fujisawa-shi (JP); Ken TOMIOKA, Yokohama-shi (JP); Hideki OBARA, Kawasaki-shi (JP)

(73) Assignee: NIKON CORPORATION, Minato-ku, Tokyo (JP)

(21) Appl. No.: 16/645,105

(22) PCT Filed: Sep. 11, 2018
(86) PCT No.: PCT/JP2018/033718

§ 371 Date: Mar. 6, 2020

(30) Foreign Application Priority Data
Sep. 11, 2017 (JP) 2017-173944

(51) Int. Cl. A61B 3/024; A61B 3/00; A61B 3/14; A61B 3/18

(52) U.S. Cl. CPC A61B 3/024; A61B 3/0008; A61B 3/14; A61B 3/0091; A61B 3/0041; A61B 3/18; A61B 3/0025

(57) ABSTRACT

An ophthalmic instrument including a light source, an optical system to guide light from the light source onto a right eye retina of a subject and/or onto a left eye retina of the subject, and a control section to control the optical system so as to perform a visual field test on the right eye retina and/or the left eye retina by the light being shone onto the right-eye retina and/or the left-eye retina.

### TECHNICAL FIELD

The technology disclosed herein relates to an ophthalmic instrument, a management device, and a method for managing an ophthalmic instrument.

### BACKGROUND ART

In the present specification ophthalmology indicates the field of medicine that handles eyes. In the present specification SLO is employed as an abbreviation to indicate a scanning laser ophthalmoscope. In the present specification OCT is employed as an abbreviation to indicate optical coherence tomography.

Japanese Patent Application Laid-Open (JP-A) No. 2016-22150 discloses a visual function examination device including an illumination optical system, a biometric information detection section, an evaluation information generation section, and a control section.

The illumination light optical system in the visual function examination device described in JP-A No. 2016-22150 includes an optical scanner disposed on the optical path of a laser beam output from a laser light source, and the laser beam that has passed through the optical scanner is shone onto the retina of a subject eye. Moreover, the biometric information detection section repetitively detects biometric information expressing the reaction of a subject to being illuminated by the laser beam. Moreover, the control section controls the illumination optical system such that an illumination intensity of the laser beam onto a single stimulation point on the retina changes monotonously while repetitively detecting the biometric information.

The evaluation information generation section in the visual function examination device described in JP-A No. 2016-22150 generates evaluation information related to the visual function of the subject eye based on the biometric information as detected. More specifically, the evaluation information generation section generates information regarding the sensitivity at a single stimulation point based on changes in the time series of the biometric information in response to the monotonous changes in the illumination intensity of the laser beam. Moreover, the evaluation information generation section generates as evaluation information a distribution of sensitivity information for plural stimulation points on the retina based on the sensitivity information generated for each of the plural stimulation points.

### SUMMARY OF INVENTION

An ophthalmic instrument according to a first aspect of technology disclosed herein includes a control device including a light source and a control section, and an eyewear terminal equipped with an optical system that includes a right-eye optical system to guide light from the light source onto a right eye retina and a left-eye optical system to guide light from the light source onto a left eye retina. The eyewear terminal and the control device are connected together by a cable including an optical fiber to supply light from the light source to the eyewear terminal. The control section executes a visual field test by controlling the optical system based on mark projection position information of plural marks for visual field test.

An ophthalmic instrument according to a second aspect of technology disclosed herein includes a control device including a control section, and an eyewear terminal equipped with an optical system to guide right-eye light that is light from a right-eye light source onto a right eye retina of a subject and to guide left-eye light that is light from a left-eye light source onto a left eye retina of the subject. The eyewear terminal and the control device are connected together by a cable including an optical fiber to supply the right-eye light from the right-eye light source and the left-eye light from the left-eye light source to the eyewear terminal. The control section transmits a control signal, to control the optical system based on the mark projection position information of plural marks for visual field test, to the eyewear terminal with the cable and executes a visual field test.

An ophthalmic instrument according to a third aspect of technology disclosed herein includes an eyewear terminal including a right-eye light source, a left-eye light source, an optical system to guide right-eye light that is light from the right-eye light source onto a right eye retina of a subject and to guide left-eye light that is light from the left-eye light source onto a left eye retina of the subject, and a control section to control the right-eye light source, the left-eye light source, and the optical system based on mark projection position information of plural marks for visual field test.

A management device according to a fourth aspect of technology disclosed herein includes a communication section to exchange data with an ophthalmic instrument, a processing section to generate transmission data for transmitting to the ophthalmic instrument by the communication section and to process received data received by the communication section, and an acquisition section to acquire examination result information representing results of a visual field test employing the ophthalmic instrument. The ophthalmic instrument includes a light source, an optical system, a control section, and a response section. The optical system includes a right-eye optical system to guide light from the light source onto a right eye retina of a subject and a left-eye optical system to guide light from the light source onto a left eye retina of the subject. The control section controls the optical system. The response section receives operation by a user of the ophthalmic instrument when the user responds to having sensed light from the light source. The transmission data includes at least instruction information to instruct which is an examination subject eye for the visual field test from out of two eyes of the subject. The received data includes at least state-of-progress information about a state of progress of the visual field test and a response signal of the response section.

A method of managing an ophthalmic instrument according to a fifth aspect of technology disclosed herein is an ophthalmic instrument management method including a step of transmitting instruction information to instruct which is an examination subject eye from out of two eyes of a subject for a visual field test employing the ophthalmic instrument, and a step of acquiring examination result information representing results of the visual field test. The ophthalmic instrument includes a control device that includes a light source, a response section, and a control section, and an eyewear terminal equipped with an optical system including a right-eye optical system to guide light from the light source onto a right eye retina and a left-eye optical system to guide light from the light source onto a left eye retina. The eyewear terminal and the control device are connected together by a cable including an optical fiber to supply light from the light source to the eyewear terminal. The control section controls the optical system. The response section receives operation by a user of the ophthalmic instrument when the user responds to having sensed light from the light source.

### BRIEF DESCRIPTION OF DRAWINGS

FIG. 1 is a schematic diagram illustrating an example of an overall configuration of an ophthalmic system according to a first exemplary embodiment.

FIG. 2 is a schematic plan view configuration diagram illustrating an example of a configuration of a wearable terminal device included in an ophthalmic system according to the first exemplary embodiment.

FIG. 3 is a block diagram illustrating an example of a hardware configuration of an electrical system of a wearable terminal device and a management device included in an ophthalmic system according to the first exemplary embodiment.

FIG. 4 is a block diagram illustrating an example of a hardware configuration of an electrical system of a server device and a viewer included in an ophthalmic system according to the first exemplary embodiment and a second exemplary embodiment.

FIG. 5 is a schematic configuration diagram illustrating an example of a configuration of a laser light source included in a wearable terminal device of an ophthalmic system according to the first exemplary embodiment.

FIG. 6 is a schematic configuration diagram illustrating an example of a configuration of an optical splitter included in a wearable terminal device of an ophthalmic system according to the first exemplary embodiment.

FIG. 7A is a flowchart illustrating an example of a flow of terminal management processing according to the first and second exemplary embodiments.

FIG. 7B is a continuation flowchart of the flowchart illustrated in FIG. 7A.

FIG. 8 is a flowchart illustrating an example of a flow of terminal management processing according to the first and second exemplary embodiments.

FIG. 9A is a flowchart illustrating an example of flow in visual field test processing included in terminal-side processing according to the first exemplary embodiment.

FIG. 9B is a continuation flowchart of the flowchart illustrated in FIG. 9A.

FIG. 10 is a flowchart illustrating an example of a flow of server-side processing according to the first and second exemplary embodiments.

FIG. 11 is a flowchart illustrating an example of a flow of display control processing according to the first and second exemplary embodiments.

FIG. 12 is a flowchart illustrating an example of a flow of communication error response processing according to the first and second exemplary embodiments.

FIG. 13 is a schematic screen layout illustrating an example of a situation in which a state-of-progress screen is displayed on a display by execution of display control processing according to the first and second exemplary embodiments.

FIG. 14 is a block diagram illustrating an example of relevant functions of an ophthalmic system according to the first exemplary embodiment.

FIG. 15 is a sequencing diagram illustrating an example of principle interactions between a wearable terminal device, a management device, a server device, and a viewer included in an ophthalmic system according to the first exemplary embodiment.

FIG. 16 is a state transition diagram illustrating an example of a comparison of a treatment flow in a hospital when an ophthalmic system according to the first exemplary embodiment is applied to plural patients against a treatment flow in a hospital when a conventional visual field test device is applied to plural patients.

FIG. 17 is a schematic plan view configuration diagram of an example of a configuration of a wearable terminal device included in an ophthalmic system according to the second exemplary embodiment.

FIG. 18 is a block diagram illustrating an example of a hardware configuration of an electrical system of a wearable terminal device and a management device included in an ophthalmic system according to the second exemplary embodiment.

FIG. 19 is flowchart illustrating an example of a flow of visual field test processing included in terminal-side processing according to the second exemplary embodiment.

FIG. 20 is a schematic diagram illustrating a modified example of an ophthalmic system according to the first and second exemplary embodiments.

FIG. 21 is a schematic diagram illustrating an example of a manner in which a terminal-side program according to the first and second exemplary embodiments is installed in a wearable terminal device.

FIG. 22 is a schematic diagram illustrating an example of a manner in which a management device-side program according to the first and second exemplary embodiments is installed in a management device.

FIG. 23 is a block diagram illustrating an example of relevant functions of an ophthalmic system according to the second exemplary embodiment.

### DESCRIPTION OF EMBODIMENTS

Explanation follows regarding examples of exemplary embodiments according to technology disclosed herein, with reference to the drawings.

First, explanation will be given regarding the meaning of the terms employed in the following description. In the following description MEMS is employed as an abbreviation to indicate micro electro mechanical systems. In the following description I/F is employed as an abbreviation to indicate an interface. In the following description I/O is employed as an abbreviation to indicate an input/output interface. In the following description USB is employed as an abbreviation to indicate a universal serial bus. In the following description ID is employed as an abbreviation to indicate identification.

In the following description CPU is employed as an abbreviation to indicate central processing unit. In the following description RAM is employed as an abbreviation to indicate random access memory. In the following description HDD is employed as an abbreviation to indicate a hard disk drive. In the following description EEPROM is employed as an abbreviation to indicate electrically erasable programmable read only memory. In the following description SSD is employed as an abbreviation to indicate a solid state drive. In the following description DVD-ROM is employed as an abbreviation to indicate digital versatile disk read only memory.

In the following description ASIC is employed as an abbreviation to indicate an application specific integrated circuit. In the following description FPGA is employed as an abbreviation to indicate a field programmable gate array. In the following description PLD is employed as an abbreviation to indicate a programmable logic device. In the following description LAN is employed as an abbreviation to indicate a local area network.

Moreover, in the present exemplary embodiments, the left and right directions indicate, for example, directions of a straight line passing through the center of the pupil of the right eye of a patient and through the center of the pupil of the left eye of the patient. Note that in the following, for ease of explanation, the “left and right directions” are referred to as the “X direction”, a direction from the center of the pupil of a subject eye toward the rear pole of the subject eye is referred to as the “Z direction”, and a direction perpendicular to both the X direction and the Z direction is referred to as the “Y direction”.

### First Exemplary Embodiment

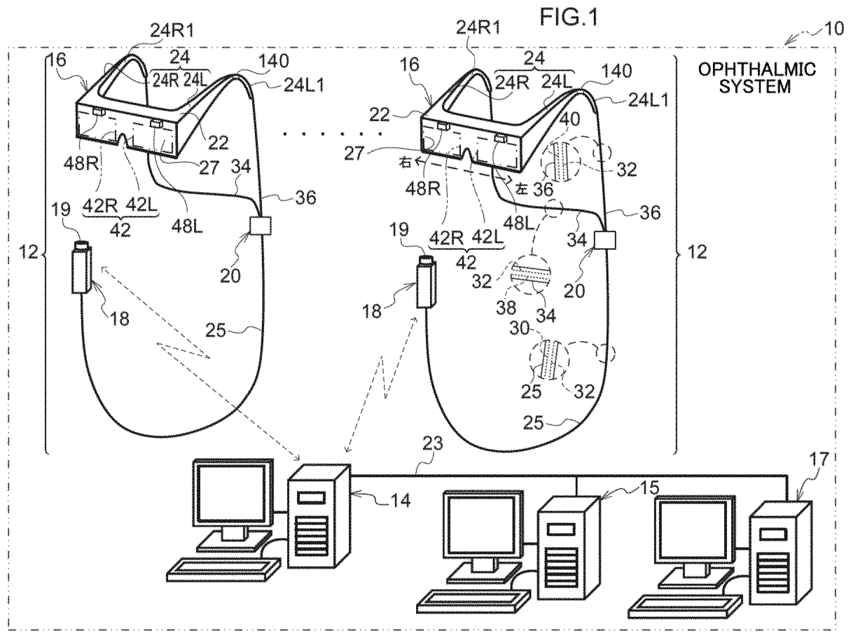

As illustrated for example in FIG. 1, an ophthalmic system 10 is a system to examine a field of view of a patient (hereafter referred simply referred to as performing a “visual field test”). In the present exemplary embodiment, the visual field test is implemented by shining a laser beam onto a retina of a subject eye of a patient (subject). Note that a laser beam is an example of “light from the light source” and of a “visual field test light that is light arising from a light source employed in visual field test” according to technology disclosed herein.

The ophthalmic system 10 includes plural wearable terminal devices 12, a management device 14, a server device 15, and a viewer 17. Note that the wearable terminal device 12 is an example of an ophthalmic instrument and of a wearable ophthalmic instrument according to technology disclosed herein.

Each of the wearable terminal devices 12 includes an eyewear terminal device 16 as an example of an eyewear terminal device according to technology disclosed herein, a control device 18, and an optical splitter 20.

The eyewear terminal device 16 is one sort of glasses-type terminal device worn by a patient. Reference here to “patient” indicates a patient having a condition of the fundus. Note that a patient is an example of a subject according to technology disclosed herein.

Similarly to ordinary glasses, the eyewear terminal device 16 includes a rim piece 22 and a temple piece 24. The eyewear terminal device 16 also includes an optical system 27.

The rim piece 22 holds the optical system 27. The temple piece 24 is broadly divided into a left temple piece 24L and a right temple piece 24R. One end portion of the left temple piece 24L is attached to a left end portion of the rim piece 22, and the right temple piece 24R is attached to the right end portion of the rim piece 22.

The left temple piece 24L includes an ear hook 24L1. The right temple piece 24R includes an ear hook 24R1. The ear hook 24L1 hooks onto the left ear of the patient, and the ear hook 24R1 hooks onto the right ear of the patient.

A speaker 140 is provided on the ear hook 24L1. The speaker 140 outputs audio under control from the control device 18. The speaker 140 may be a speaker that directly imparts a sound wave to the eardrum of the patient, or may be a bone conduction speaker that indirectly transmits vibrations to the eardrum of the patient. The speaker 140 is an example of a notification section to notify information to the patient by activating the hearing of the patient.

The control device 18 is, for example, employed by being grasped by the patient, or by being worn by the patient on their clothes or on their person. The control device 18 is equipped with a response button 19. The response button 19 is an example of a response section according to technology disclosed herein. The response button 19 referred to here is merely an example thereof, and the technology disclosed herein is not limited thereto. For example, a touch panel may be employed instead of the response button 19, or a microphone may be employed to pick up speech of a patient in response to the patient sensing the laser beam and a speech recognition device may be employed to recognize the audio picked up by the microphone. In such cases the touch panel and the speech recognition device output response information, described later, in response to activation by the patient.

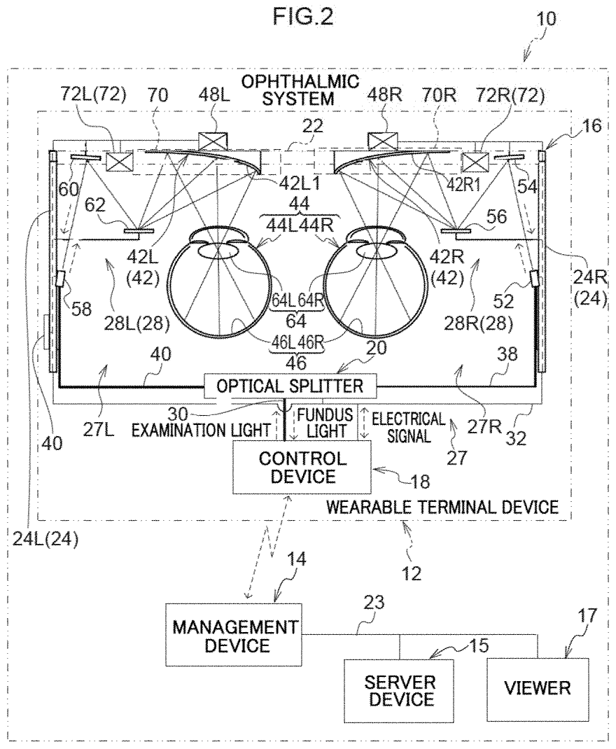

The response button 19 is operated by the patient and outputs information according to operation by the patient. The response button 19 receives an operation as to whether or not the patient has sensed the laser beam when the laser beam was shone onto a retina 46 (see FIG. 2) of a subject eye 44 (see FIG. 2). In other words, the response button 19 receives operation by the patient in cases in which the patient responds to having sensed the laser beam. Namely, processing is performed to associate the response information of the response button with mark projection position information.

The response button 19 is also sometimes pressed by the patient when the patient responds to a question from a medical service professional. Note that reference here to a “medical service profession” indicates, for example, a medical technician in ophthalmology with the qualifications of an orthoptist who performs vision examinations under instruction from an ophthalmologist. The response button 19 and the control device 18 are connected together either by wire and/or wirelessly so as to enable communication therebetween, and response information arising from operation of the response button 19 is transmitted to the control device 18. One response button 19 is associated with the control device 18 by a number, such as a machine number. Examples of wireless communication include communication by Wi-Fi (registered trademark), Bluetooth (registered trademark), or the like. Examples of wired communication include communication using a cable.

The control device 18 is connected to the management device 14 so as to be capable of wireless communication therewith, and the control device 18 exchanges various kinds of information with the management device 14. The control device 18 is connected to the optical splitter 20 by a cable 25 and controls the optical splitter 20. The control device 18 may also be connected to the management device 14 in a state capable of wireless communication therewith.

The cable 25 includes an optical fiber 30 and a bus line 32. The control device 18 supplies a laser beam to the optical splitter 20 through the optical fiber 30 and controls the optical splitter 20 through the bus line 32.

The optical system 27 is equipped with the optical splitter 20. The optical splitter 20 is connected to the eyewear terminal device 16 by cables 34, 36. The cable 34 is connected to the right temple piece 24R, and the cable 36 is connected to the left temple piece 24L. The cables 34, 36 both include the bus line 32. Thus the control device 18 exchanges various kinds of electrical signal with the eyewear terminal device 16 through the bus line 32.

The cable 34 includes an optical fiber 38, and the cable 36 includes an optical fiber 40. The optical splitter 20 splits the laser beam supplied from the control device 18 through the optical fiber 30 so that a laser beam passes into the optical fiber 38 and/or into the optical fiber 40. One of the laser beams obtained by splitting with the optical splitter 20 is supplied into the eyewear terminal device 16 through the optical fiber 38. The other of the laser beams obtained by splitting with the optical splitter 20 is supplied into the eyewear terminal device 16 through the optical fiber 40.

The optical system 27 includes a reflection mirror 42. The reflection mirror 42 is an example of a reflection member according to technology disclosed herein. The reflection mirror 42 guides laser beams onto the retinas 46 of the subject eyes 44 of the patient by reflecting the laser beam supplied from the optical splitter 20 through the cables 34, 36, as illustrated for example in FIG. 2. Note that the subject eyes 44 are broadly composed of a right eye 44R and a left eye 44L, as illustrated for example in FIG. 2. The retinas 46 are broadly composed of a retina 46R that is an example of a right retina according to technology disclosed herein, and a retina 46L that is an example of a left retina according to technology disclosed herein.

The reflection mirrors 42 are broadly composed of a right-eye reflection mirror 42R and a left-eye reflection mirror 42L. The right-eye reflection mirror 42R is held by the rim piece 22 so as to be positioned in front of the right eye 44R of the patient when the eyewear terminal device 16 is in a correctly worn state. The left-eye reflection mirror 42L is held by the rim piece 22 so as to be positioned in front of the left eye 44L of the patient when the eyewear terminal device 16 is in a correctly worn state.

The right-eye reflection mirror 42R guides a laser beam onto the retina 46R of the right eye 44R of the patient by reflecting the laser beam supplied from the optical splitter 20 through the optical fiber 38, as illustrated for example in FIG. 2. The left-eye reflection mirror 42L guides a laser beam onto the retina 46L of the left eye 44L of the patient by reflecting the laser beam supplied from the optical splitter 20 through the optical fiber 40, as illustrated for example in FIG. 2.

The eyewear terminal device 16 is equipped with a right-eye inward-facing camera 48R and a left-eye inward-facing camera 48L. The right-eye inward-facing camera 48R and the left-eye inward-facing camera 48L image an imaging subject under control from the control device 18.

The right-eye inward-facing camera 48R and the left-eye inward-facing camera 48L are attached to an upper edge of the rim piece 22. The right-eye inward-facing camera 48R is provided at a position shifted away from the right-eye reflection mirror 42R in the Y direction, and images the anterior segment of the right eye 44R as an imaging subject from diagonally above a region in front of the right eye 44R. The left-eye inward-facing camera 48L is provided at a position shifted away from the left-eye reflection mirror 42L in the Y direction, and images the anterior segment of the left eye 44L as an imaging subject from diagonally above a region in front of the left eye 44L. The right-eye inward-facing camera 48R and the left-eye inward-facing camera 48L are examples of anterior segment cameras according to technology disclosed herein. Moreover, although the right-eye inward-facing camera 48R and the left-eye inward-facing camera 48L are given as examples here, the technology disclosed herein is not limited thereto. For example, instead employing the right-eye inward-facing camera 48R and the left-eye inward-facing camera 48L, a single camera may be employed to image both the anterior segment of the right eye 44R and the anterior segment of the left eye 44L.

The management device 14 performs unified management of visual field tests performed by each of the plural wearable terminal devices 12. The visual field tests by the wearable terminal devices 12 referred to here are, in other words, visual field tests being performed using the wearable terminal devices 12. Management of the visual field tests referred to here indicates, for example, management including management of the laser beams employed in visual field test, and management of sensing it formation expressing visual sensing by the patients of illuminated laser beams achieved by shining the laser beams onto the retinas 46.

The control device 18 supplies laser beams into the eyewear terminal device 16 through the optical fibers 30, 38, 40 under instruction from the management device 14.

Note that although explanation has been given in the present exemplary embodiment of an example in which wireless communication is performed between the wearable terminal devices 12 and the management device 14, technology disclosed herein is not limited thereto. For example, wired communication may be performed between the wearable terminal devices 12 and the management device 14.

The server device 15 provides information and/or performs information processing in response to requests from external devices such as from the management device 14 and/or the viewer 17 etc., and performs unified management of personal information of plural patients. The server device 15 is connected to the management device 14 through a cable 23 and exchanges various kinds of information with the management device 14. An example of the cable 23 is a LAN cable. Note that although wired communication is performed between the server device 15 and the management device 14 in the present exemplary embodiment, technology disclosed herein is not limited thereto, and wireless communication may be performed between the server device 15 and the management device 14.

The optical system 27 guides the laser beams onto the retina 46R and/or the retina 46L, as illustrated for example in FIG. 2. The optical system 27 includes a scanner 28 and the reflection mirror 42. The scanner 28 scans laser beams supplied from the control device 18 through the optical splitter 20. The reflection mirror 42 reflects the laser beams being scanned by the scanner 28 onto the retinas 46.

The optical system 27 includes a right-eye optical system 27R and a left-eye optical system 27L. The optical splitter 20 splits the laser beam supplied from the control device 18 through the optical fiber 30 so as to pass into the right-eye optical system 27R and the left-eye optical system 27L.

The tight-ere optical system 27R guides the laser beam being supplied from the optical splitter 20 through the optical fiber 38 onto the retina 46R. The left-eye optical system 27L guides the laser beam being supplied from the optical splitter 20 through the optical fiber 40 onto the retina 46L.

The scanner 28 includes a right-eye scanner 28R and a left-eye scanner 28L. The right-eye optical system 27R includes the right-eye scanner 28R and the right-eye reflection mirror 42R. The left-eye optical system 27L includes the left-eye scanner 28L and the left-eye reflection mirror 42L.

The right-eye scanner 28R includes MEMS mirrors 54, 56 and the right-eye reflection mirror 42R, and scans the laser beam supplied from the optical splitter 20 through the optical fiber 38. A right-eye illumination section 52 shines the laser beam supplied from the optical splitter 20 through the optical fiber 38. The MEMS mirror 54 is disposed on the direction the laser beam is shone in by the right-eye illumination section 52, and the MEMS mirror 54 reflects the laser beam being shone from the right-eye illumination section 52 so as to be guided onto the MEMS mirror 56. The MEMS mirror 56 reflects the laser beam guided by the MEMS mirror 54 so as to be guided onto the right-eye reflection mirror 42R.

For example, the MEMS mirror 54 scans the laser beam in the Y direction, and the MEMS mirror 56 scans the laser beam in the X direction. Two-dimensional scanning on the retina is enabled by the MEMS mirrors 54, 56, enabling an image to be two-dimensionally scanned and projected onto the retina.

Obviously a configuration may be adopted in which the MEMS mirror 54 scans in the X direction and the MEMS mirror 56 scans in the Y direction.

Furthermore, the right-eye scanner 28R may be configured by employing the reflection mirror 42R and a MEMS mirror 56 capable of scanning in the XY directions.

The right-eye reflection mirror 42R reflects the laser beam scanned by the right-eye scanner 28R onto the retina 46R.

The right-eye reflection mirror 42R includes a curved surface 42R1. The curved surface 42R1 is a surface formed so as to be concave as viewed from the right eye 44R of the patient in a state in which the eyewear terminal device 16 is being worn. Due to the laser beam guided by the MEMS mirror 56 being reflected at the curved surface 42R1, the laser beam is guided through a crystalline lens 64R behind the pupil of the right eye 44R and onto the retina 46R of the right eye 44R.

The left-eye scanner 28L includes MEMS mirrors 60, 62 and the left-eye reflection mirror 42L, and scans the laser beam supplied from the optical splitter 20 through the optical fiber 40. A left-eye illumination section 58 shines a laser beam supplied from the optical splitter 20 through the optical fiber 40. The MEMS mirror 60 is disposed on the direction of illumination of the laser beam by the left-eye illumination section 58, and the MEMS mirror 60 reflects the laser beam shone from the left-eye illumination section 58 so as to be guided onto the MEMS mirror 62. The MEMS mirror 62 reflects the laser beam guided by the MEMS mirror 60 so as to be guided onto the left-eye reflection mirror 42L.

For example, the MEMS mirror 60 scans the laser beam in the Y direction, and the MEMS mirror 62 scans the laser beam in the X direction. Two-dimensional scanning on the retina is enabled by the MEMS mirrors 60, 62, enabling an image to be two-dimensionally scanned and projected onto the retina.

Obviously a configuration may be adopted in which the MEMS mirror 60 scans in the X direction and the MEMS mirror 62 scans in the Y direction.

Furthermore, the left-eye scanner 28L may be configured by employing the reflection mirror 42L and a MEMS mirror 56 capable of scanning in the XY directions.

Although the MEMS mirrors 54, 56, 60, 62 are given as examples in the example illustrated in FIG. 2, the technology disclosed herein is not limited thereto. For example, instead of the MEMS mirrors 54, 56, 60, 62, or together with one or more of the MEMS mirrors 54, 56, 60, 62, a mirror such as a galvanometer mirror and/or a polygon mirror or the like that enables electrical control of the position on the reflection face may be employed.

The left-eye reflection mirror 42L reflects the laser beam scanned by the left-eye scanner 28L onto the retina 46L.

The left-eye reflection mirror 42L includes a curved surface 42L1. The curved surface 42L1 is a surface formed so as to be concave as viewed from the left eye 44L of the patient in a state in which the eyewear terminal device 16 is being worn. Due to the laser beam guided by the MEMS mirror 62 being reflected at the curved surface 42L1, the laser beam is guided through a crystalline lens 64L behind the pupil of the left eye 46R and onto the retina 46L of the left eve 44L.

Note that when there is no need to discriminate between the crystalline lenses 64R, 64L in the description below, for ease of explanation they will be referred to as “crystalline lenses 64”.

The optical system 27 includes a right-eye sliding mechanism 70R, a left-eye sliding mechanism 70L, a right-eye drive source 72R, and a left-eye drive source 72L. Examples of the right-eye drive source 72R and the left-eye drive source 72L include a stepping motor, a solenoid, and a piezoelectric element or the like. Note that when there is no need to discriminate between the right-eye drive source 72R and the left-eve drive source 72L in the description below, for ease of explanation they will be referred to as “mirror drive sources 72”.

The right-eye sliding mechanism 70R is attached to the rim piece 22, and is held thereby so as to enable the right-eye reflection mirror 42R to slide in the left-right direction. The right-eye sliding mechanism 70R is connected to the right-eye drive source 72R, and slides the right-eye reflection mirror 42R in the left-right direction on receipt of motive force generated by the right-eye drive source 72R.

The left-eye sliding mechanism 70L is attached to the rim piece 22, and is held thereby so as to enable the left-eye reflection mirror 42L to slide in the left-right direction. The left-eye sliding mechanism 70L is connected to the left-eye drive source 72L, and slides the left-eye reflection mirror 42L in the left-right direction on receipt of motive force generated by the left-eye drive source 72L.

In the ophthalmic system 10 according to the present exemplary embodiment, an image arising from the laser beam is projected onto the retina 46 of the subject eye 44 by a Maxwellian view optical system. Reference here to “Maxwellian view optical system” indicates an optical system in which laser beams are converged by the crystalline lenses 64 behind the pupils of the subject eyes 44, and images arising from the laser beams are projected onto the retinas 46 of the subject eyes 44 by the laser beams converged by the crystalline lenses 64 being shone onto the retinas 46 of the subject eyes 44. In the ophthalmic system 10 according to the present exemplary embodiment, the Maxwellian view optical system is implemented by the scanner 28 and the mirror drive sources 72 being controlled by the control device 18.

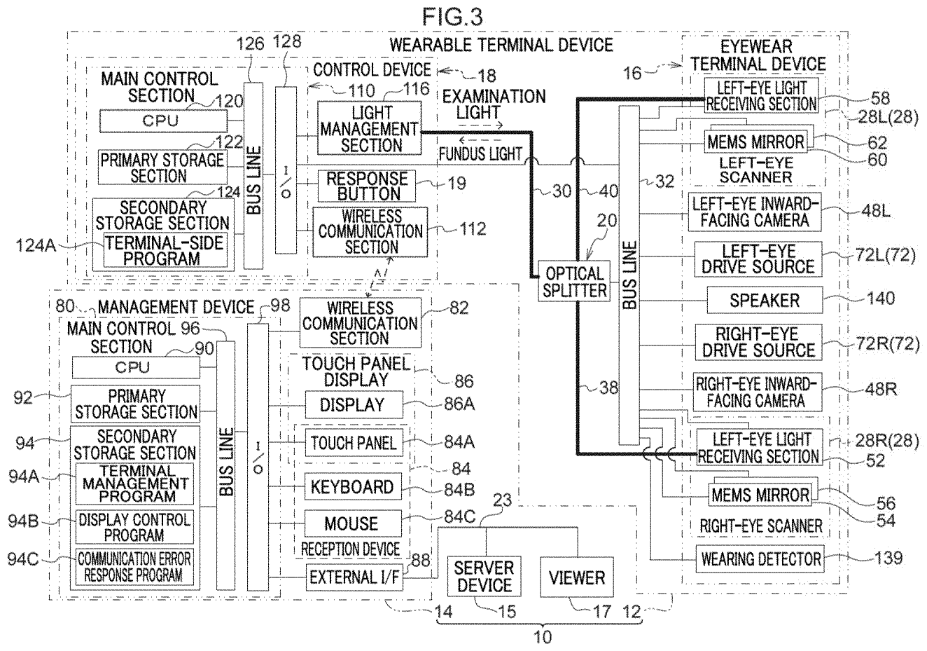

As illustrated for example in FIG. 3, the management device 14 includes a main control section 80, a wireless communication section 82, a reception device 84, a touch panel display 86, and an external I/F 88.

The main control section 80 includes a CPU 90, a primary storage section 92, a secondary storage section 94, a bus line 96, and an I/O 98. The CPU 90, the primary storage section 92, and the secondary storage section 94 are connected together through the bus line 96. The I/O 98 is connected to the bus line 96. Note that although a single CPU is employed for the CPU 90 in the present exemplary embodiment, plural CPUs may be employed instead of the CPU 90.

The CPU 90 controls the management device 14 overall. The primary storage section 92 is volatile memory employed as a work area or the like when various programs are being executed. An example of the primary storage section 92 is RAM. The secondary storage section 94 is non-volatile memory to store a program and various parameters and the like employed to control the basic operation of the management device 14. Examples of the secondary storage section 94 include a HDD, EEPROM, and flash memory or the like.

The wireless communication section 82 is connected to the I/O 98. The CPU 90 outputs to the wireless communication section 82 an electrical signal for transmission to the control device 18. The wireless communication section 82 transmits the electrical signal input from the CPU 90 to the control device 18 using radio waves. The wireless communication section 82 also receives radio waves from the control device 18, and outputs to the CPU 90 an electrical signal according to the received radio waves.

A wireless communication section 112 is an example of a communication section according to technology disclosed herein. Namely, the wireless communication section 82 transmits to the wearable terminal device 12 control information that is control information for the wearable terminal device 12 to control the wearable terminal device 12, and that includes instruction information to instruct which of the two eyes of the patient is the examination subject eye for ophthalmic examination.

The reception device 84 includes a touch panel 84A, a keyboard 84B, and a mouse 84C, with the touch panel 84A, the keyboard 84B, and the mouse 84C being connected to the I/O 98. This accordingly enables the CPU 90 to ascertain various instructions received by each of the touch panel 84A, the keyboard 84B, and the mouse 84C.

The external I/F 88 is connected to external devices, such as the server device 15, a personal computer, and/or a USB memory or the like, and is employed to exchange various information between the external devices and the CPU 90. In the example illustrated in FIG. 3, the external I/F 88 is connected to the server device 15 by the cable 23.

The touch panel display 86 includes a display 86A and a touch panel 84A. The display 86A is an example of a display section according to technology disclosed herein. The display 86A is connected to the I/O 98 and displays various information including images under control from the CPU 90. The touch panel 84A is a transparent touch panel superimposed on the display 86A.

The secondary storage section 94 stores a terminal management program 94A, a display control program 94B, and a communication error response program 94C. When there is no need to discriminate in the description between the terminal management program 94A, the display control program 94B, and the communication error response program 94C below, for ease of explanation they be referred to as “management device-side programs”.

The CPU 90 reads the management device-side programs from the secondary storage section 94, and expands the read management device-side programs into the primary storage section 92. The CPU 90 executes the management device-side programs that have been expanded into the primary storage section 92.

The control device 18 is equipped with, as well as the response button 19 mentioned above, a main control section 110, the wireless communication section 112, a laser light source 114 [not in FIG. 3], and a light source control circuit 116.

The main control section 110 includes a CPU 120, a primary storage section 122, a secondary storage section 124, a bus line 126, and an I/O 128. The CPU 120, the primary storage section 122, and the secondary storage section 124 are connected together through the bus line 126. The I/O 128 is connected to the bus line 126. Note that although a single CPU is employed for the CPU 120 in the present exemplary embodiment, plural CPUs may be employed instead of the CPU 120.

The CPU 120 controls the wearable terminal device 12 overall. The primary storage section 122 is volatile memory employed as a work area or the like when various programs are being executed. An example of the primary storage section 122 is RAM. The secondary storage section 124 is non-volatile memory to store a program and various parameters and the like employed to control the basic operation of the wearable terminal device 12. Examples of the secondary storage section 124 include a HDD, EEPROM, and flash memory or the like.

The response button 19 is connected to the I/O 128, and a response signal is output from the response button 19 to the CPU 120 when the response button 19 is pressed.

The wireless communication section 112 performs wireless communication with the management device 14 to allow the management device 14 to manage the visual field test performed by the wearable terminal device 12. The wireless communication section 112 is connected to the I/O 128. The CPU 120 outputs to the wireless communication section 112 an electrical signal for transmission to the management device 14. The wireless communication section 112 transmits the electrical signal input from the CPU 120 to the management device 14 using radio waves. The wireless communication section 112 also receives radio waves from the management device 14, and outputs to the CPU 120 an electrical signal according to the received radio waves.

The laser light source 114 is connected to the optical splitter 20 through the optical fiber 30. The laser light source 114 generates a laser beam, and the generated laser beam is emitted to the optical splitter 20 through the optical fiber 30.

The laser light source 114 is connected to the light source control circuit 116. The light source control circuit 116 is connected to the I/O 128. The light source control circuit 116 supplies light source control signals to the laser light source under instruction from the CPU 120, and controls the laser light source 114.

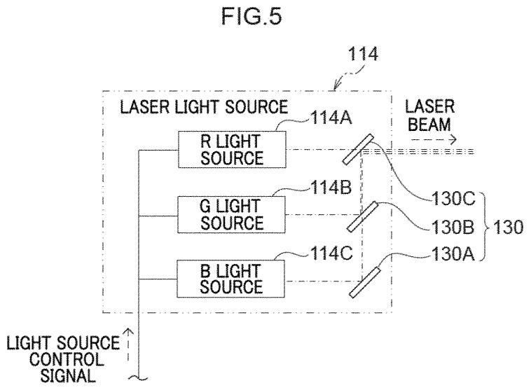

As illustrated in the example in FIG. 5, the laser light source 114 includes a R light source 114A, a G light source 114B, a B light source 114C, and a mirror unit 130.

The R light source 114A emits an R laser beam that is an R laser beam from out of R (red), G (green), and B (blue). The G light source 114B emits a G laser beam that is a G laser beam from out of R, G, and B. The B light source 114C emits a B laser beam that is a B laser beam from out R, G, and B. Note that although an example is given here in which the laser light source 114 is equipped with the R light source 114A, the G light source 114B, and the B light source 114C, technology disclosed herein is not limited thereto. For example, the laser light source 114 may be equipped with an IR light source (not illustrated in the drawings). “IR” is employed here as an abbreviation for “near-infrared”. Such an IR light source emits near-infrared light that is a laser beam employed in SLO and/or OCT ID imaging.

The mirror unit 130 is equipped with a first mirror 130A, a second mirror 130B, and a third mirror 130C. From out of the first mirror 130A, the second mirror 130B, and the third mirror 130C, the second mirror 130B is a dichroic mirror that transmits the B laser beam while reflecting the G laser beam. The third mirror 130C is also a dichroic mirror, and transmits the R laser beam while reflecting the G laser beam and the B laser beam.

The first mirror 130A is disposed in the direction in which the B laser beam is emitted by the B light source 114C, and guides the B laser beam to the second mirror 130B reflecting the B laser beam emitted from the B light source 114C.

The second mirror 130B is disposed in the direction in which the G laser beam is emitted by the G light source 114B and is also on the direction of progression of the B laser beam reflected by the first mirror 130A. The second mirror 130B guides the G laser beam to the first mirror 130A by reflecting the G laser beam emitted from the G light source 114B, and also guides the B laser beam to the first mirror 130A by transmitting the B laser beam reflected by the first mirror 130A.

The third mirror 130C is disposed on the direction in which the R laser beam is emitted by the R light source 114A and also on the direction of progression of the G laser beam reflected by the second mirror 130B as well as on the direction of progression of the G laser beam transmitted through the second mirror 130B. The third mirror 130C transmits the R laser beam emitted from the R light source 114A. The third mirror 130C externally emits the R laser beam, the G laser beam, and the B laser beam by reflecting the G laser beam and the B laser beam so as to travel in the same direction as the R laser beam. In the present exemplary embodiment, for ease of explanation the R laser beam, the G laser beam, and the B laser beam emitted externally from the laser light source 114 are simply referred to as “laser beam”.

As illustrated for example in FIG. 3, the bus line 32 is connected to the I/O 128, and the optical splitter 20 is connected to the bus line 32. Thus the optical splitter 20 acts under the control of the CPU 120.

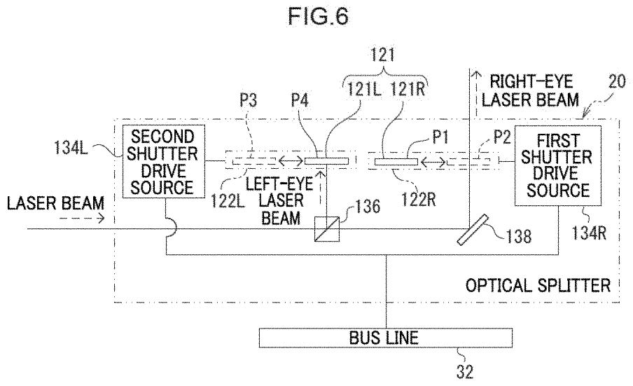

In the example illustrated in FIG. 6, the optical splitter 20 includes a right-eye shutter 121R, a left-eye shutter 121L, a first sliding mechanism 122R, a second sliding mechanism 122L, a first shutter drive source 134R, a second shutter drive source 134L, a beam splitter 136, and a reflection mirror 138.

When there is no need to discriminate between the right-eye shutter 121R and the left-eye shutter 121L in the description below, for ease of explanation they will be referred to as “shutters 121”.

The beam splitter 136 both reflects and transmits the laser beam supplied from the laser light source 114 through the optical fiber 130. The left-eye laser beam that is the laser beam reflected by the beam splitter 136 proceeds toward the inlet port of the optical fiber 40 (see FIG. 1 and FIG. 2).

The reflection mirror 138 reflects the laser beam transmitted through the beam splitter 136. The right-eye laser beam that is the laser beam reflected by the reflection mirror 138 proceeds toward the inlet port of the optical fiber 38 (see FIG. 1 and FIG. 2).

The first sliding mechanism 122R holds the right-eye shutter 121R so as to be capable of sliding between a first position P1 and a second position P2. The first position P1 indicates a position where the right-eye laser beam is transmitted and guided into the inlet port of the optical fiber 38, and the second position P2 indicates a position where the right-eye laser beam is blocked.

The second sliding mechanism 122L holds the left-eye shutter 121L so as to be capable of sliding between a third position P3 and a fourth position P4. The third position P3 indicates a position where the left-eye lase beam is transmitted and guided into the inlet port of the optical fiber 40, and the fourth position P4 indicates a position where the left-eye laser beam is blocked.

Examples of the first shutter drive source 134R and the second shutter drive source 134L include a stepping motor, a solenoid, and a piezoelectric element or the like. The first shutter drive source 134R and the second shutter drive source 134L are connected to the bus line 32, and the first shutter drive source 134R and the second shutter drive source 134L operate under the control of the CPU 120.

The first sliding mechanism 122R is connected to the first shutter drive source 134R, and slides the right-eye shutter 121R between the first position P1 and the second position P2 on receipt of motive force generated by the first shutter drive source 134R.

The second sliding mechanism 122L is connected to the second shutter drive source 134L and slides the left-eye shutter 121L between the third position P3 and the fourth position P4 on receipt of motive force generated by the second shutter drive source 134L.

In the example illustrated in FIG. 6, the right-eye laser beam is supplied into the optical fiber 38 by the right-eye shutter 121R being disposed at the first position P1, and the left-eye laser beam is blocked by the left eye shutter 121L by the left-eye shutter 121L being disposed at the fourth position P4.

For example, as illustrated in FIG. 3, the speaker 140 is connected to the bus line 32 and outputs audio under the control of the CPU 120.

The right-eye drive source 72R and the left-eye drive source 72L are connected to the bus line 32, and the CPU 120 controls the right-eye drive source 72R and the left-eye drive source 72L.

The right-eye inward-facing camera 48R and the left-eye inward-facing camera 48L are connected to the bus line 32, and the CPU 120 exchanges various kinds of information with the left-eye inward-facing camera 48L and the right-eye inward-facing camera 48R.

The right-eye illumination section 52, the left-eye illumination section 58, and the MEMS mirrors 54, 56, 60, 62 are also connected to the bus line 32, and the CPU 120 controls the right-eye illumination section 52, the left-eye illumination section 58, and the MEMS mirrors 54, 56, 60, 62.

A wearing detector 139 is connected to the bus line 32. The wearing detector 139 is, for example, a pressure sensor. The wearing detector 139 is provided on the frame of the eyewear terminal device 16 and detects whether the eyewear terminal device 16 is being worn correctly. The CPU 120 acquires a detection result from the wearing detector 139. The frame of the eyewear terminal device 350 indicates, for example, the rim piece 22 and the temple piece 24.

The secondary storage section 124 stores a terminal-side program 124A. The CPU 120 reads the terminal-side program 124A from the secondary storage section 124, and expands the read terminal-side program 124A into the primary storage section 122. The CPU 120 executes the terminal-side program 124A that has been expanded into the primary storage section 122.

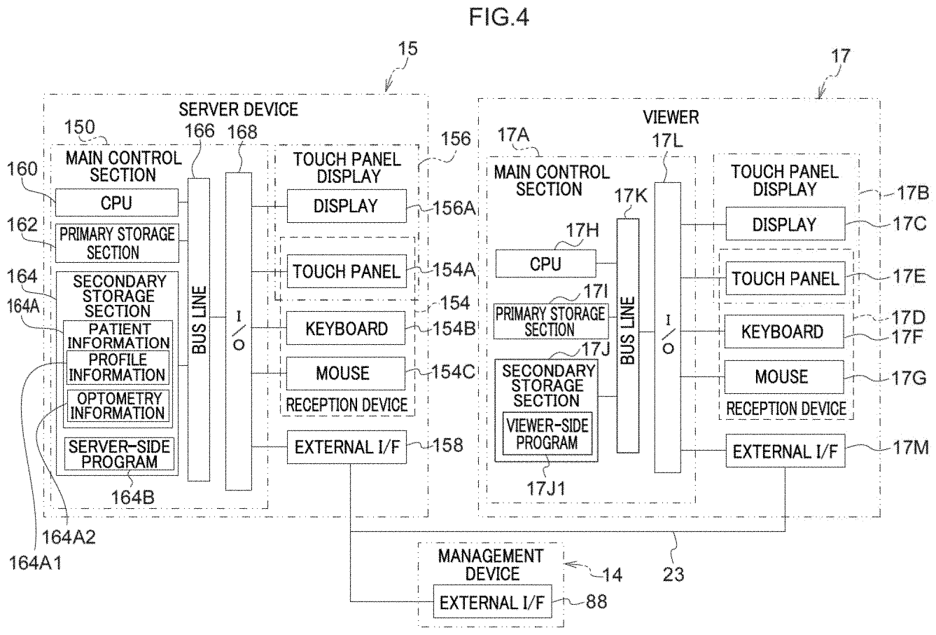

As illustrated in the example of FIG. 4, the server device 15 is equipped with a main control section 150, a reception device 154, a touch panel display 156, and an external I/F 158.

The main control section 150 includes a CPU 160, a primary storage section 162, a secondary storage section 164, a bus line 166, and an I/O 168. The CPU 160, the primary storage section 162, and the secondary storage section 164 are connected together through the bus line 166. The I/O 168 is connected to the bus line 166. Note that although a single CPU is employed for the CPU 160 in the present exemplary embodiment, plural CPUs may be employed instead of the CPU 160.

The CPU 160 controls the server device 15 overall. The primary storage section 162 is volatile memory employed as a work area or the like when various programs are being executed. An example of the primary storage section 162 is RAM. The secondary storage section 164 is non-volatile memory to store a program and various parameters and the like employed to control the basic operation of the server device 164. Examples of the secondary storage section 164 include a HDD, EEPROM, and flash memory or the like.

The reception device 154 includes a touch panel 154A, a keyboard 154B, and a mouse 154C, with the touch panel 154A, the keyboard 154B, and the mouse 154C being connected to the I/O 168. This accordingly enables the CPU 160 to ascertain various instructions received by each of the touch panel 154A, the keyboard 154B, and the mouse 154C.

The external I/F 158 is connected to external devices, such as the management device 14, a personal computer, and/or a USB memory or the like, and is employed to exchange various information between the external devices and the CPU 160. In the example illustrated in FIG. 3, the external I/F 158 is connected to the external I/F 88 of the management device 14 by the cable 23.

The touch panel display 156 includes a display 156A and a touch panel 154A. The display 86A is connected to the I/O 168 and displays various information including images under control from the CPU 160. The touch panel 154A is a transparent touch panel superimposed on the display 156A.

The secondary storage section 164 stores patient information 164A and a server-side program 164B.

The patient information 164A is information related to the patient. In the present exemplary embodiment, the patient information 164A includes patient profile information 164A1 (for example, an ID to identify the patient, patient name, patient gender, patient age, physical information, past treatment history, current patient information such as hospitalization status, risk of disease, and physical state and the like) and optometry information 164A2 of optometry performed on the patient. The optometry information 164A2 includes other information related to the left eye/right eye of the patient (for example, conical refractive power, corneal wavefront aberration, visual acuity, myopia/hyperopia/astigmatism, field of view, eye axial length, fundus photograph or the like that is information obtained with a different ophthalmic instrument). Examples of the different ophthalmic instrument include a refractive power measurement instrument, eye axial length measurement instrument, a visual acuity detector, an anterior segment measurement instrument, a posterior segment measurement instrument, and the like.

As illustrated for example in FIG. 4, the viewer 17 is equipped with a main control section 17A, a touch panel display 17B, a reception device 17D, and an external I/F 17M.

The main control section 17A includes a CPU 17H, a primary storage section 17I, a secondary storage section 17J, a bus line 17K, and an I/O 17L. The CPU 17H is connected to the primary storage section 17I, and the secondary storage section 17J through the bus line 17K. The I/O 17L is connected to bus line 17K. Note that although a single CPU is employed for the CPU 17H in the present exemplary embodiment, plural CPUs may be employed instead of the CPU 17H.

The CPU 17H controls the viewer 17 overall. The primary storage section 17I is non-volatile memory employed as a work area or the like when various programs are being executed. An example of the primary storage section 17I is RAM. The secondary storage section 17J is non-volatile memory employed to store a program and various parameters and the like employed to control the basic operation of the viewer 17. Examples of the secondary storage section 17J include a HDD, EEPROM, and flash memory or the like. The secondary storage section 164 stores a viewer-side program 17J1.

The reception device 17D includes a touch panel 17E, a keyboard 17F, and a mouse 17G, and the touch panel 17E, the keyboard 17F, and the mouse 17G are connected to the I/O 17L. This accordingly enables the CPU 17H to ascertain various instructions received through the touch panel 17E, the keyboard 17F, or the mouse 17G.

The external I/F 17M is connected to external devices, such as the management device 14, the server device 15, a personal computer, and/or USB memory or the like, and is employed to exchange of various information between the external devices and the CPU 17H. Note that in the example illustrated in FIG. 4, the external I/F 17M is connected to the external I/F 88 of the management device 14 and the external I/F 158 of the server device 15 by the cable 23.

The touch panel display 17B includes a display 17C and a touch panel 17E. The display 17C is connected to the I/O 17L and displays various information including images under the control of the CPU 17H. The touch panel 17E is a transparent touch panel superimposed on the display 17C.

The CPU 160 reads the server-side program 164B from the secondary storage section 164 and expands the read server-side program 164B into the primary storage section 162. The CPU 160 executes the server-side program 164B that has been expanded into the primary storage section 162.

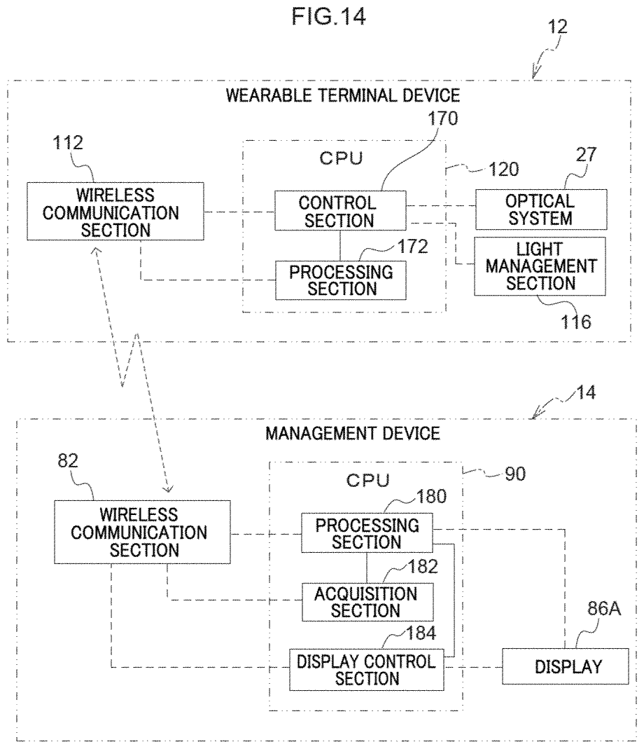

By executing the terminal-side program 124A, the CPU 120 of the main control section 110 included in the wearable terminal device 12 operates as a control section 170 and a processing section 172, as illustrated in the example of FIG. 14.

The processing section 172 performs processing required to cause the CPU 120 to operate as the control section 170. The control section 170 controls the optical system 27 so as perform a visual field test on the retina 46R and/or the retina 46L by the laser beams being shone onto the retina 46R and/or the retina 46L.

The processing section 172 serves as an example of a first processing section according to technology disclosed herein and performs processing according to operation of the response button 19. The processing. according to operation of the response button 19 is, for example, processing to store mark projection position information, described later, in the primary storage section 122, and/or processing to output sensory information according to a response signal input through the response button 19. The sensory information indicates information expressing that the patient has visually sensed the laser beam.

Moreover, the processing section 172 serves as an example of a second processing section according to technology disclosed herein and performs processing to transmit information related to the state of progress of visual field test. The destination for transmission of the information related to the state of progress of visual field test is, for example, the management device 14, however the technology disclosed herein is not limited thereto. For example, a configuration may be adopted in which the information related to the state of progress of visual field test is transmitted to an external device other than the management device 14, such as a personal computer, and/or a server device or the like.

By executing the terminal management program 94A, the CPU 90 of the main control section 80 included in the management device 14 operates as a processing section 180 and an acquisition section 182, as illustrated in the example in FIG. 14. By executing the display control program 94B, the CPU 90 operates as a processing section 180 and display control section 184, as illustrated in the example in FIG. 14.

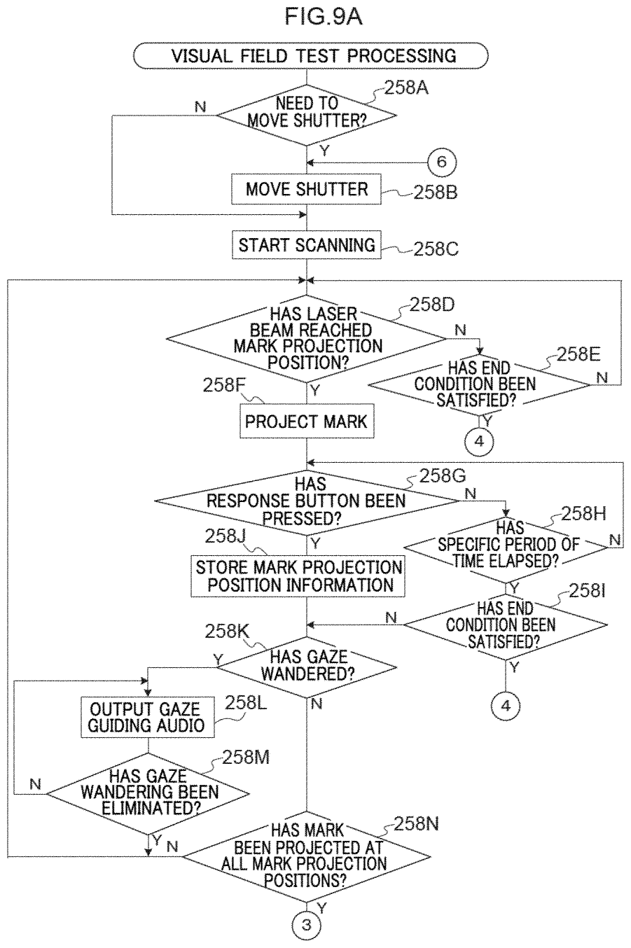

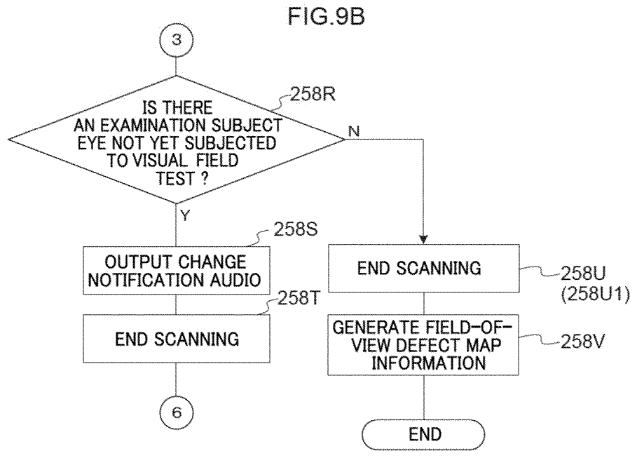

The processing section 180 performs processing required to cause the CPU 90 to operate as the acquisition section 182 and the display control section 184. The acquisition section 182 acquires examination result information representing the results of the visual field test. Examples of the examination result information include field of view defect map information, described later (see step 258V of FIG. 9B).

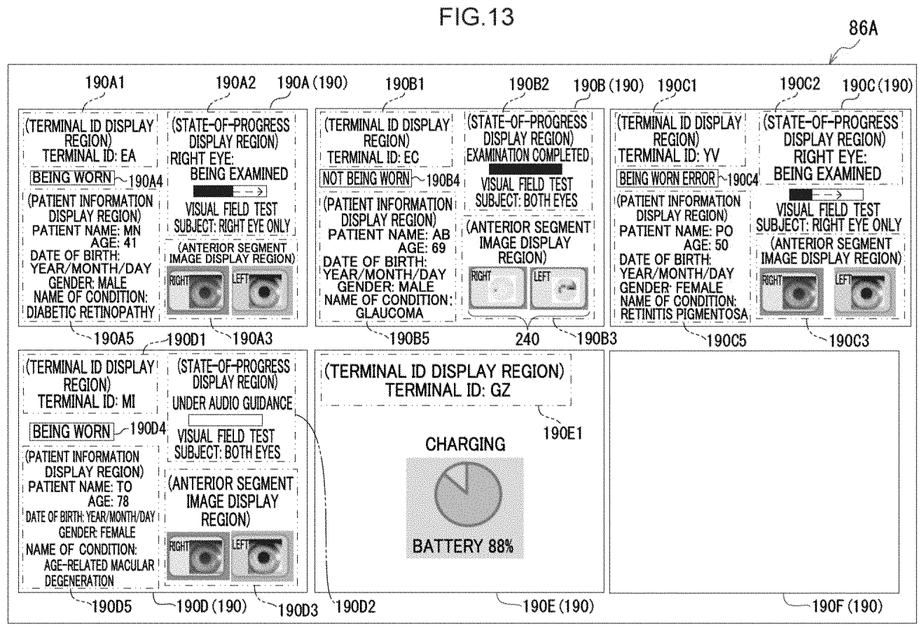

The display control section 184 generates a state-of-progress screen 190 (see FIG. 13) that is a screen representing the state of progress of visual field test, and outputs an image signal representing an image including the generated state-of-progress screen 190. The display 86A displays the state-of-progress screen 190 based on the image signal input from the display control section 184. Namely, the display control section 184 controls the display 86A so as to cause the display 86A to display the state-of-progress screen 190. The display control section 184 acquires from the wearable terminal device 12 state-of-progress information indicating the state of progress of visual field test by the wearable terminal device 12 and the management device 14 communicating through the wireless communication sections 82, 112. The display control section 184 generates the state-of-progress screen 190 based on the state-of-progress information, and controls the display 86A so that the generated state-of-progress screen 190 is displayed on the display 86A.

Note that, as illustrated in the example of FIG. 13, in the present exemplary embodiment the state-of-progress screen 190 is broadly composed of a first state-of-progress screen 190A, a second state-of-progress screen 190B, a third state-of-progress screen 190C, a fourth state-of-progress screen 190D, a fifth state-of-progress screen 190E, and a sixth state-of-progress screen 190F. Namely, the first state-of-progress screen 190A, the second state-of-progress screen 190B, the third state-of-progress screen 190C, the fourth state-of-progress screen 190D, the fifth state-of-progress screen 190E, and the sixth state-of-progress screen 190F are displayed on the display 86A.

Explanation next follows regarding operation of the sections of the ophthalmic system 10 according to technology disclosed herein.

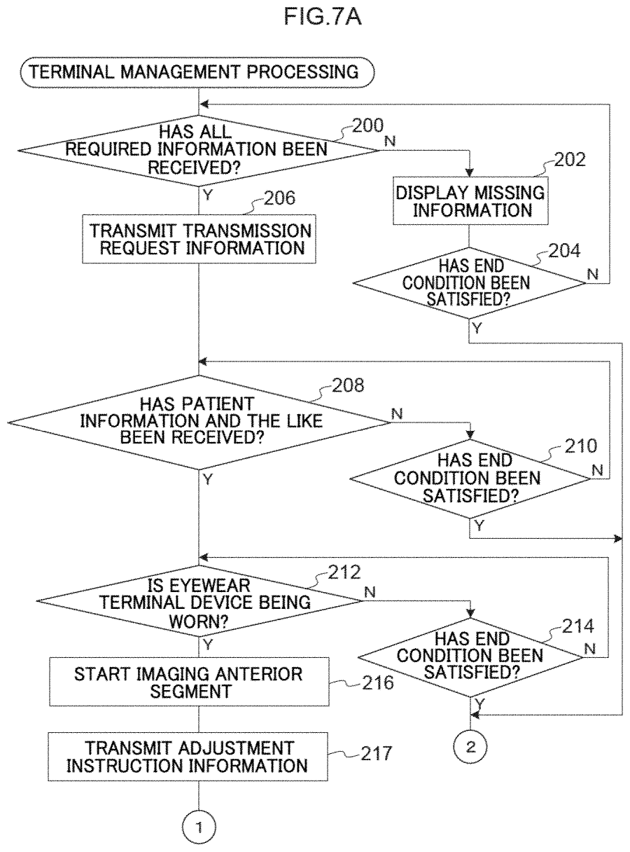

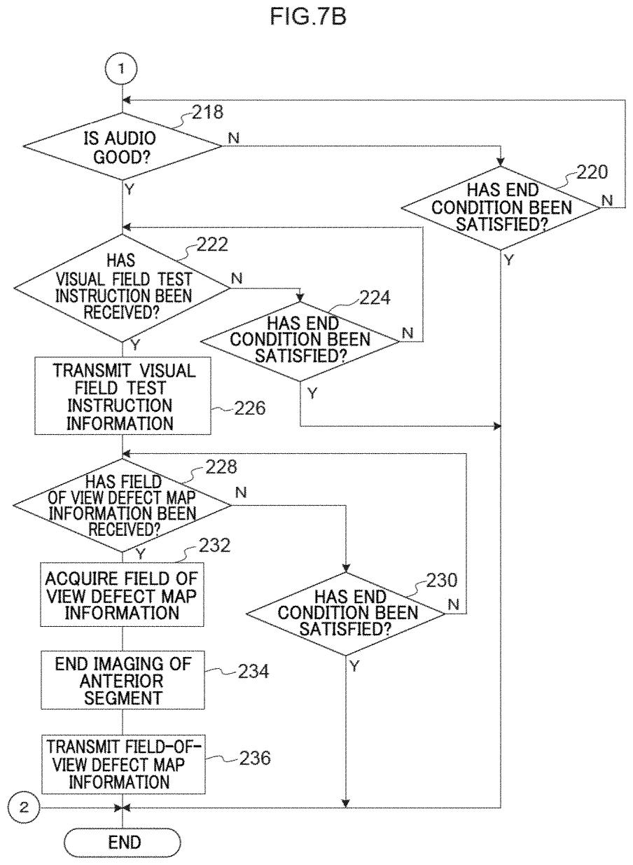

First explanation will be given regarding terminal management processing implemented by the CPU 90 executing the terminal management program 94A when an instruction to start executing of terminal management processing is received by the reception device 84, with reference to FIG. 7A and FIG. 7B.

For ease of explanation, the following description assumes that at least one patient is appropriately wearing one of the wearable terminal devices 12.

Moreover, for ease of explanation, the following description assumes that a fixation target is being presented in a visible state to the patient.

In the terminal management processing illustrated in FIG. 7A first, at step 200, determination is made as to whether or not the processing section 180 has received all of the required information required by the reception device 84 and/or the server device 15. The “required information” indicates information required for an ophthalmic examination, such as examination subject eye instruction information, patient ID, eyewear ID, and the like. The examination subject eye instruction information refers to information instructing which the subject eye 44 subjected to examination is from out of the right eye 44R and the left eye 44L (namely, information indicating whether the examination subject is the right eye 44R, the left eye 44L, or both eyes). The patient ID indicates information enabling the patient to be uniquely identified. The eyewear ID indicates information enabling the wearable terminal device 12 being worn by the patient to be uniquely identified.

Processing transitions to step 202 when negative determination is made at step 200, i.e. when not all of the required information has been received by the reception device 84. Processing transitions to step 206 when affirmative determination is made at step 200, i.e. when all of the required information has been received by the reception device 84.

At step 202 the processing section 180 displays missing information on the display 86A, and then processing transitions to step 204. The missing information indicates, for example, a message showing which information is missing from out of the information required for ophthalmic examination.

At step 204, the processing section 180 determines whether or not the processing section 180 has satisfied an end condition relating to terminal management processing. The end condition relating to terminal management processing indicates a condition to end the terminal management processing. Examples of the end condition relating to terminal management processing include a condition that a specific period of time has elapsed, a condition that an end instruction has been received by the reception device 84, and/or a condition that a situation requiring the terminal management processing to be forcibly ended has been detected by the CPU 90.

Processing transitions to step 200 when negative determination is made at step 204, i.e. when the end condition relating to terminal management processing has not been satisfied. The terminal management processing is ended when affirmative determination is made at step 204, i.e. when the end condition relating to terminal management processing has been satisfied.

At step 206, the processing section 180 transmits to the server device 15 transmission request information requesting the patient information 164A to be transmitted, and then processing transitions to step 208.

By executing the processing of the present step 206, the patient information and the like is transmitted from the server device 15 by the processing of step 256 included in the server-side processing, described later. The patient information and the like indicates information including at least the patient information 164A.

At step 208, the processing section 180 determines whether or not the patient information and the like has been received by the wireless communication section 82. Processing transitions to step 210 when negative determination is made at step 208, i.e. when the patient information and the like has not been received. Processing transitions to step 212 when affirmative determination is made at step 206, i.e. when the patient information and the like has been received.

At step 210 the processing section 180 determines whether or not the end condition relating to terminal management processing has been satisfied. Processing transitions to step 208 when negative determination is made at step 210, i.e. when the end condition relating to terminal management processing has not been satisfied. The terminal management processing is ended when affirmative determination is made at step 210, i.e. when the end condition relating to terminal management processing has been satisfied.

At step 212, the processing section 180 determines whether or not the eyewear terminal device 16 is being worn correctly by the patient by communicating with the control device 18 through the wireless communication sections 82, 112. Processing transitions to step 214 when negative determination is made at step 212, i.e. when the eyewear terminal device 16 is not being worn correctly by the patient. Processing transitions to step 215 when affirmative determination is made at step 212, i.e. when the eyewear terminal device 15 is being worn correctly by the patient. Note that whether or not the eyewear terminal device 16 is being worn correctly by the patient is determined based on detection results by the wearing detector 139.

At step 214 the processing section 180 determines whether or not the end condition relating to terminal management processing has been satisfied. Processing transitions to step 212 when negative determination is made at step 214, i.e. when the end condition relating to terminal management processing has not been satisfied. The terminal management processing is ended when affirmative determination is made at step 214, i.e. when the end condition relating to terminal management processing has been satisfied.

At step 216, the processing section 180 causes the right-eye inward-facing camera 48R and the left-eye inward-facing camera 48L to start imaging the anterior segment of the subject eye 44 by performing wireless communication with the control device 18, and then processing transitions to step 217.

In the following, for ease of explanation, an image obtained by imaging the anterior segment of the right eye 44R with the right-eye inward-facing camera 48R is referred to as a right-eye anterior segment image, and an image obtained by imaging the anterior segment of the left eye 44L with the left-eye inward-facing camera 48L is referred to as a left-eye anterior segment image. When there is no need to discriminate between the right-eye anterior segment image and the left-eye anterior segment image in the description below, for ease of explanation they will be referred to simply as “anterior segment images”.

Note that in the present exemplary embodiment the anterior segment of the left eye 44L is imaged by the left-eye inward-facing camera 48L, and the anterior segment of the right eye 44R is imaged by the right-eye inward-facing camera 48R at the frame rate of 60 fps (frames/second). Namely a video image is acquired with the anterior segment of the subject eye 44 as the imaging subject by the processing section 180 causing the left-eye inward-facing camera 48L and the right-eye inward-facing camera 48R to operate.

At step 217, the processing section 217 transmits adjustment instruction information to the wearable terminal device 12, and then processing transitions to step 218. Adjustment instruction information indicates here information to instruct the wearable terminal device 12 to adjust the position of the reflection mirror 42, to correct the laser beam optical axis, and to perform home positioning.

At step 218 (see FIG. 7B), the processing section 180 causes test audio to be output by the speaker 140 by performing wireless communication with the control device 18, and determines whether or not the audio of the speaker 140 is good. The test audio indicates, for example, audio of “PLEASE PRESS THE RESPONSE BUTTON WHEN YOU HEAR A SOUND” or the like. Thus, for example, whether or not the audio of the speaker 140 is good is determined by whether or not the response button 19 is pressed by the patient while the test audio is being output from the speaker 140.

Processing transitions to step 220 when negative determination is made at step 218, i.e. when the audio of the speaker 140 is not good. Processing transitions to step 222 when affirmative determination is made at step 218, i.e. when the audio of the speaker 140 is good.

At step 220, the processing section 180 determines whether or not the end condition relating to terminal management processing has been satisfied. Processing transitions to step 218 when negative determination is made at step 220, i.e. when the end condition relating to terminal management processing has not been satisfied. The terminal management processing is ended when affirmative determination is made at step 220, i.e. when the end condition relating to terminal management processing has been satisfied.

At step 222, the processing section 180 determines whether or not a visual field test instruction has been received by the reception device 84. The visual field test instruction indicates an instruction to cause the wearable terminal devices 12 to execute visual field test processing, described later.

Processing transitions to step 224 when negative determination is made at step 222, i.e. when the visual field test instruction has not yet been received by the reception device 84. Processing transitions to step 226 when affirmative determination is made at step 222, i.e. when the visual field test instruction has been received by the reception device 84.

At step 224, the processing section 180 determines whether or not the end condition relating to terminal management processing has been satisfied. Processing transitions to step 222 when negative determination is made at step 224, i.e. when the end condition relating to terminal management processing has not been satisfied. The terminal management processing is ended when affirmative determination is made at step 224, i.e. when the end condition relating to terminal management processing has been satisfied.

At step 226, the processing section 180 transmits the visual field test instruction information of an example of the technology disclosed herein to the wearable terminal device 12, and then processing transitions to step 228. Note that the visual field test instruction information indicates information to instruct the wearable terminal device 12 to execute visual field test processing (FIG. 9A and FIG. 9B), described later. The visual field test instruction information includes required information required to be received at step 200, and patient information and the like received by the wireless communication section 82 at step 208.

In the present exemplary embodiment, mark projection position information of plural marks for visual field test is incorporated in the terminal-side program 124A. The mark projection position information indicates information representing positions where marks are to be projected onto the retinas 46 (hereafter also referred to as “mark projection positions” or “projection positions”). The “marks” referred to here indicate, for example, marks sensed as white dots for normal retinas 46. The projection of the marks onto the retinas 46 is implemented by shining the laser beams.

Information indicating the brightness (intensity) of the laser beams may be combined with the mark projection position information, with the mark projection position information held for use in the visual field test. Combining the information about the projection position and the brightness enables information about the sensitivity of the retina to be obtained in visual field test.

Moreover, the mark projection position information of the plural marks in the terminal-side program 124A is employed by the control section 170 of the control device 18 to control the scanner 28. Namely, the laser beams are shone onto the positions (projection positions according to the mark projection position information) represented by the mark projection position information of the plural marks due to the scanner 28 being controlled by the control section 170 according to the mark projection position information of the plural marks.

At step 228, the acquisition section 182 determines whether or not field-of-view defect map information transmitted from the wearable terminal device 12 has been received by the wireless communication section 82. Note that the field-of-view defect map information is transmitted from the wearable terminal device 12 by the processing of step 260 included in terminal-side processing, described later, being executed by the processing section 172.

Processing transitions to step 230 when negative determination is made at step 228, i.e. when the field-of-view defect imp information transmitted from the wearable terminal device 12 is not received by the wireless communication section 82. Processing transitions to step 232 when affirmative determination is made at step 228, i.e. when the field-of-view defect map information transmitted from the wearable terminal device 12 has been received by the wireless communication section 82.

At step 230, the processing section 180 determines whether or not the end condition relating to terminal management processing has been satisfied. Processing transitions to step 228 when negative determination is made at step 230, i.e. when the end condition relating to terminal management processing has not been satisfied. The terminal management processing is ended when affirmative determination is made at step 230, i.e. when the end condition relating to terminal management processing has been satisfied.

At step 232, the acquisition section 182 acquires the field-of-view defect map information received by the wireless communication section 82 at step 228, and then processing transitions to step 234.

At step 234, the processing section 180 causes the right-eye inward-facing camera 48R and the left-eye inward-facing camera 48L to end imaging of the anterior segments of the subject eyes 44 by performing wireless communication with the control device 18, then processing transitions to step 236.

At step 236, the processing section 180 transmits the field-of-view defect map information acquired by the acquisition section 182 at step 232 to the server device 15, and then ends the terminal management processing. Note that the display content of the state-of-progress screen 190 illustrated in FIG. 13 is updated as appropriate by the processing section 180 and the display control section 184 of the management device 14, based on the information transmitted from the wearable terminal device 12, and the state-of-progress screen 190 with updated display content is displayed on the display 86A.

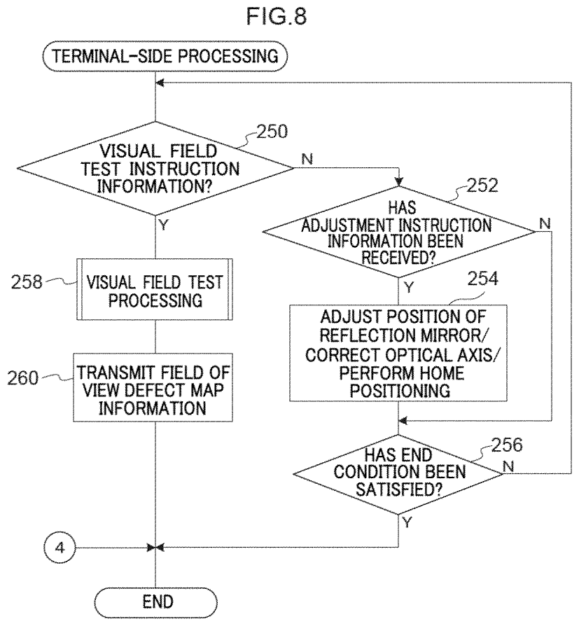

Next, explanation follows regarding the terminal-side processing implemented by the CPU 120 executing the terminal-side program 124A when the main power source (not illustrated in the drawings) for the wearable terminal device 12 is turned on, with reference to FIG. 8.

In the terminal-side processing illustrated in FIG. 8, the processing section 172 determines at step 250 whether or not the visual field test instruction information from the management device 14 has been received by the wireless communication section 112. Processing transitions to step 252 when negative determination is made at step 250, i.e. when the visual field test instruction information from the management device 14 has not been received by the wireless communication section 112. Processing transitions to step 258 when affirmative determination is made at step 250, i.e. when the visual field test instruction information from the management device 14 has been received by the wireless communication section 112.

At step 252, the processing section 172 determines whether or not the adjustment instruction information, transmitted from the management device 14 by execution of the processing of step 217 included in the terminal management processing, has been received by the wireless communication section 112. Processing transitions to step 256 when negative determination is made at step 252, i.e. when the adjustment instruction information has not been received by the wireless communication section 112. Processing transitions to step 254 when affirmative determination is made at step 252, i.e. when the adjustment instruction information has been received by the wireless communication section 112.

Processing transitions to step 256 after the control section 170 has, at step 254, performed adjustment of the position of the reflection mirror 42, correction of the optical axes of the laser beams, and home positioning.

In order to adjust the position of the reflection mirror 42, correct the optical axes of the laser beams, and perform home positioning at step 254, first the inter-pupil distance is detected by the control section 170 based on the latest right-eye anterior segment image and the latest left-eye anterior segment image. Then, the adjustment of the position of the reflection mirror 42, correction of the optical axes of the laser beams, and home positioning is performed by the control section 170 based on the eyewear ID of the wearable terminal device 12, the detected inter-pupil distance, and the like. Note that the inter-pupil distance referred to here indicates the distance between the pupil in the anterior segment of the right eye 44R as represented in the right-eye anterior segment image and the pupil in the anterior segment of the left eye 44L as represented in the left-eye anterior segment image. Moreover, the position of the reflection mirror 42 is adjusted by the mirror drive sources 72 being controlled by the control section 170. The correction of the optical axes of the laser beams and the home positioning is implemented by the scanner 28 being controlled by the control section 170.

At step 256, the processing section 171 determines whether or not the end condition relating to terminal-side processing has been satisfied. The end condition relating to terminal-side processing indicates a condition to end the terminal-side processing. Examples of the end condition relating to terminal-side processing include a condition that a specific period of time has elapsed, a condition that information has been received indicating that an end instruction from the management device 14, and/or a condition that a situation requiring the terminal-side processing to be forcibly ended has been detected by the CPU 120.

Processing transitions to step 250 when negative determination is made at step 256, i.e. when the end condition relating to terminal-side processing has not been satisfied. The terminal-side processing is ended when affirmative determination is made at step 256, i.e. when the end condition relating to terminal-side processing has been satisfied.

At step 258, the control section 170 executes visual field test processing as illustrated in the example of FIG. 9A and FIG. 9B, and then processing transitions to step 260.

As illustrated in the example of FIG. 9A, at step 258A in the visual field test processing, the control section 170 determines whether or not a shutter 121 needs to be moved based on the examination subject eye instruction information in the previously mentioned required information included in the visual field test instruction information.

Processing transitions to step 258C when negative determination is made at step 258A, i.e. when there is no need to move the shutter 121. Processing transitions to step 258B when affirmative determination is made at step 258A, i.e. when there is a need to move the shutter 121.

At step 258B, the control section 170 moves the shutter 121 based on the examination subject eye instruction information in the previously mentioned required information included in the visual field test instruction information, and then processing transitions to step 258C.

At step 258C, the control section 170 causes a light management section 114 and the optical system 27 to start scanning the laser beam over the retina 46 of the examination subject eye, and then processing transitions to step 258D.

At step 258D, the control section 170 determines whether or not the laser beam has reached the position indicated by the mark projection position information for one mark out of the mark projection position information for plural marks in the terminal-side program 124A. At the present step 258D, the same mark projection position information is reused as the “mark projection position information for one mark” when this follows from affirmative determination being made at step 258M. Moreover, at the present step 258D, mark projection position information for an unused mark from out of the mark projection position information for plural marks is used as the “mark projection position information for one mark” when this follows from negative determination being made at step 258N.

In the present exemplary embodiment, although the sequence in which the mark projection position information for plural marks is used at the present step 258D is predetermined, the technology disclosed herein is not limited thereto. For example, mark projection position information instructed by a medical service professional via the management device 14 may be used at the present step 258D. Moreover, the sequence in which the mark projection position information is used at the present step 258D may be changeable by the medical service professional via the management device 14.

Processing transitions to step 258E when negative determination is made at step 258D, i.e. when the laser beam has not reached the position indicated by the mark projection position information for one mark out of the mark projection position information for plural marks in the terminal-side program 124A. Process transitions to step 258F when affirmative determination is made at step 258D, i.e. when the laser, beam has reached the position indicated by the mark projection position information for one mark out of the mark projection position information of the plural marks.

At step 258E, the control section 170 determines whether or not the end condition relating to terminal-side processing has been satisfied. Processing transitions to step 258D when negative determination is made at step 258E, i.e. when the end condition relating to terminal-side processing has not been satisfied. The terminal-side processing is ended when affirmative determination is made at step 258E, i.e. when the end condition relating to terminal-side processing has been satisfied.

At step 258F, the control section 170 projects the mark onto the retina 46 by controlling a laser light source unit 113 through a light source control circuit 115, and then processing transitions to step 258G. Note that the position where the mark is projected is a position indicated by the latest mark projection position information employed at step 258D.

At step 258G, the control section 170 determines whether or not the response button 19 has been pressed. Whether or not the response button 19 has been pressed is determined by whether or not a response signal has been input from the response button 19.

Processing transitions to step 258H when negative determination is made at step 258G, i.e. when the response button 19 has not been pressed. Processing transitions to step 258J when affirmative determination is made at step 258G, i.e. when the response button 19 has been pressed.

At step 258J, the control section 170 stores the latest mark projection position information in the primary storage section 122, and then processing transitions to step 258K. The latest mark projection position information referred to here indicates the latest mark projection position information used at step 258D, in other words indicates the mark projection position information for the mark being projected onto the retina 46 at the timing when the response button 19 was pressed.

At step 258H, the control section 170 determines whether or not a predetermined period of time (for example, 2 seconds) has elapsed from when the processing of step 258F was executed. Processing transitions to step 258G when negative determination is made at step 258H, i.e. when the predetermined period of time has not elapsed from when the processing of step 258F was executed. Processing transitions to step 258I when affirmative determination is made at step 258H, i.e. when the predetermined period of time has elapsed from when the processing of step 258F was executed.

At step 258I, the control section 170 determines whether or not the end condition relating to terminal-side processing has been satisfied. Processing transitions to step 258K when negative determination is made at step 258I, i.e. when the end condition relating to terminal-side processing has not been satisfied. The terminal-side processing is ended when affirmative determination is made at step 258I, i.e. when the end condition relating to terminal-side processing has been satisfied.

At step 258K, the control section 170 determines whether or not the gaze of the patient has wandered from the fixation target. The determination as to whether or not the gaze of the patient has wandered from the fixation target is determined based on the latest anterior segment image.

Processing transitions to step 258L when affirmative determination is made at step 258K, i.e. when the gaze of the patient has wandered from the fixation target. Processing transitions to step 258N when negative determination is made at step 258K, i.e. when the gaze of the patient has not wandered from the fixation target.

At step 258L, the control section 170 causes the speaker 140 to output gaze guiding audio, and then processing transitions to step 258M.

The gaze guiding audio indicates audio to guide the gaze in a direction toward the fixation target. The gaze guiding audio is generated according to the positional relationship between the gaze and the fixation target. The position of the gaze may be identified based on the latest anterior segment image. Examples of the gaze guiding audio include audio content of “PLEASE LOOK AT THE FIXATION TARGET”, audio content of “A LITTLE BIT MORE TO THE RIGHT, PLEASE”, etc.

At step 258M, the control section 170 determines whether or not wandering of the gaze of the patient from the fixation target has been eliminated. Determination as to whether or not wandering of the gaze of the patient from the fixation target has been eliminated is determined based on the latest anterior segment image.

Processing transitions to step 258L when negative determination is made at step 258M, i.e. when wandering of the gaze of the patient from the fixation target has not been eliminated. Processing transitions to step 258D when affirmative determination is made at step 258M, i.e. when wandering of the gaze of the patient from the fixation target has been eliminated.

At step 258N, the control section 170 determines whether or not marks have been projected onto all of the mark projection positions. Processing transitions to step 258D when negative determination is made at step 258N, i.e. when marks have not yet been projected onto all of the mark projection positions. Processing transitions to step 258R of FIG. 9B when affirmative determination is made at step 258N, i.e, when marks have been projected onto all of the mark projection positions.

At step 258R, the control section 170 determines whether or not there is still an examination subject eye that has not yet been subjected to the visual field test. The determination as to whether or not there is still an examination subject eye that has not yet been subjected to the visual field test is determined based on the examination subject eye instruction information in the previously mentioned required information included in the visual field test instruction information.

Processing transitions to step 258S when affirmative determination is made at step 258R, i.e. when there is still an examination subject eye that has not yet been subjected to the visual field test. Processing transitions to step 258U when negative determination is made at step 258R, i.e. when there is not an examination subject eye that has not yet been subjected to the visual field test.

At step 258S, the control section 170 causes change notification audio to be output by the speaker 140, and then processing transitions to step 258T. The change notification audio indicates audio to notify the patient of a change to the examination subject eye. An example of the change notification audio is audio content of “THE VISUAL FIELD TEST FOR THE RIGHT EYE IS NOW COMPLETE AND THE VISUAL FIELD TEST WILL NOW BE PERFORMED ON THE LEFT EYE”.

At step 258T, the control section 170 controls the light management section 114 and the optical system 27 so as to cause the light management section 114 and the optical system 27 to stop scanning of the laser beam on the retina 46 of the examination subject eye, and then processing transitions to step 258B.

At step 258U, the control section 170 controls the light management section 114 and the optical system 27 so as to cause the light management section 114 and the optical system 27 to stop scanning of the laser beam on the retina 46 of the examination subject eye, and then processing transitions to step 258V.

At step 258V, the control section 170 generates field-of-view defect map information based on the mark projection position information stored in the primary storage section 122 by executing the processing of step 258J, and then ends the visual field test processing.

Note that the field-of-view defect map information indicates information including the patient ID, information to draw a field-of-view defect map, an image of a field-of-view defect map, and the like. The field-of-view defect map indicates a map enabling the identification of defective sites in the field of view of the patient. A field-of-view defect map 240 is displayed in an image display region 190B3 of the second state-of-progress screen 190B illustrated in FIG. 13. In the field-of-view defect map 240, defective sites and normal sites are represented by the tone of a gray scale, with the principle defective sites being displayed in black.

At step 260 in FIG. 8, the processing section 172 transmits the field-of-view defect map information, generated by executing the processing of step 258V (see FIG. 9B) included in the visual field test processing, to the management device 14 through the wireless communication section 112, and then ends the terminal-side processing.

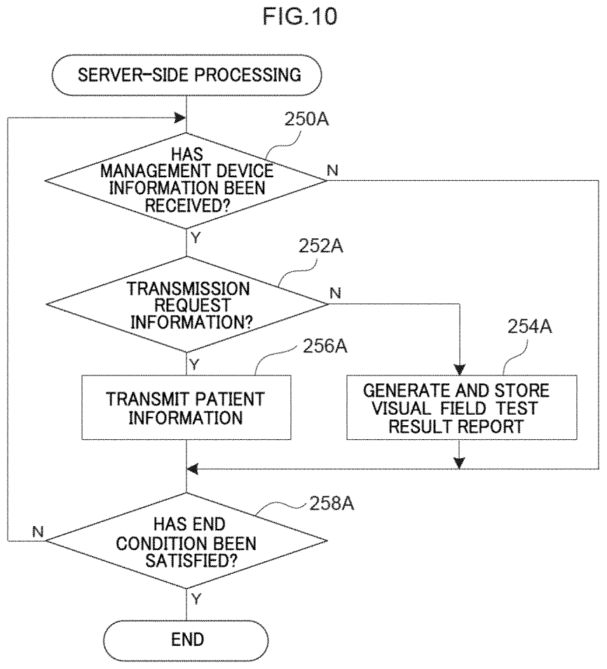

Next, explanation follows regarding server-side processing implemented by the CPU 160 executing the server-side program 164B when power is turned on to a main power source (not illustrated in the drawings) of the server device 15, with reference to FIG. 10.

In the server-side processing illustrated in FIG. 10, the CPU 160 first determines at step 250A whether or not management device information has been received. The management device information indicates information transmitted to the server device 15 by the terminal management processing being executed by the CPU 90 of the management device 14.

Processing transitions to step 258A when negative determination is made at step 250A, i.e. when the management device information has not been received. Processing transitions to step 252A when affirmative determination is made at step 250A, i.e. when the management device information has been received.

At step 252A, the CPU 160 determines whether or not the management device information received at step 250A is the transmission request information. Processing transitions to step 254A when negative determination is made at step 252A, i.e. when the management device information received at step 250A is not the transmission request information, namely, when the management device information received at step 250A is the field-of-view defect map information. Processing transitions to step 256A when affirmative determination is made at step 252A, i.e. when the management device information received at step 250A is the transmission request information.

At step 254A, the CPU 160A generates a visual field test result report that is a report to indicate the results of the visual field test based on the field-of-view defect map information, stores the generated visual field test result report in the secondary storage section 164, and then processing transitions to step 258A. The generated visual field test result report is, for example, transmitted to an external device, such as the viewer 17 or the like when requested by the viewer 17 or the like.

At step 256A, the CPU 160 transmits the patient information and the like described above to the management device 14, and then processing transitions to step 258A. The patient information 164A included in the patient information and the like is acquired from the secondary storage section 164.

At step 258A, the CPU 160 determines whether or not the end condition relating to server-side processing has been satisfied. The end condition relating to server-side processing indicates a condition to end the server-side processing. Examples of the end condition relating to server-side processing include a condition that a specific period of time has elapsed, a condition that the reception device 154 has received an end instruction, and/or a condition that a situation requiring the server-side processing to be forcibly ended has been detected by the CPU 160.

Processing transitions to step 250A when negative determination is made at step 258A, i.e. when the end condition relating to server-side processing has not been satisfied. The server-side processing is ended when affirmative determination is made at step 258A, i.e. when the end condition relating to server-side processing has been satisfied.

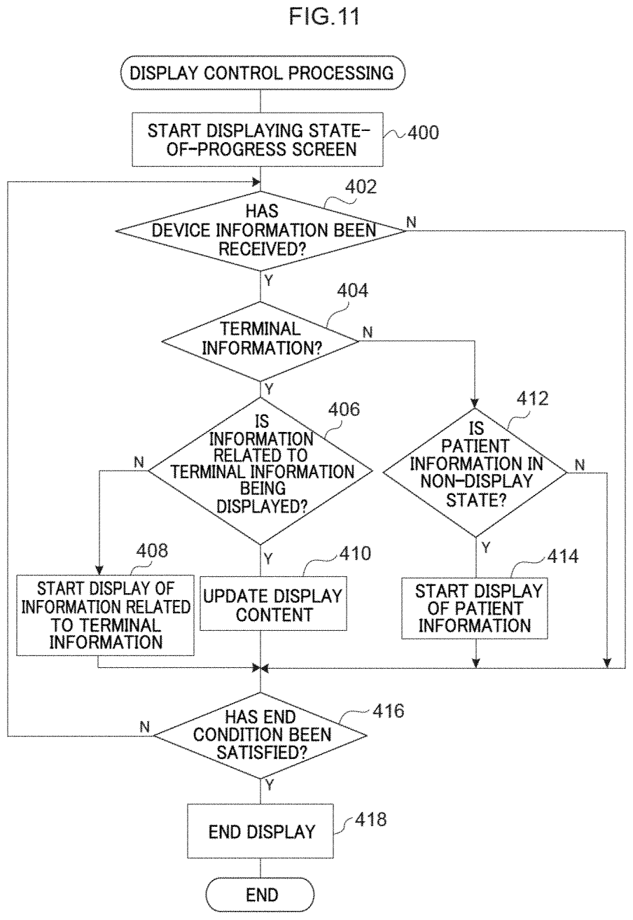

Explanation next follows regarding the display control processing implemented by the CPU 90 executing the display control program 94B by starting to execute the terminal management processing, with reference to FIG. 11.

In the following description, for ease of explanation, all of the required information will be assumed to have been received by the reception device 84 through the execution of the processing of step 200 included in the terminal management processing illustrated in FIG. 7A.

Moreover, in the following description, for ease of explanation, the management device 14 will be assumed to be capable of managing a maximum of six of the wearable terminal devices 12. Note that six devices is merely an example of the number of devices, and configurations that have various maximum numbers of manageable devices may be adopted. Furthermore, in the following, for ease of explanation, an example will be described in which there is an assumption that a state of communication has been established between the management device 14 and five of the wearable terminal devices 12, and that the display control processing is for one of the wearable terminal devices 12 from out of these five wearable terminal devices 12.

At step 400 of the display control processing illustrated in FIG. 11, the display control section 184 causes the display 86A to start to display the state-of-progress screen 190, as illustrated in the example of FIG. 13, and then processing transitions to step 402.

At step 402, the display control section 184 determines whether or not the device information has been received. Reference here to “device information” indicates terminal information transmitted from the processing section 171 of the wearable terminal device 12 through the wireless communication section 112 by communication performed with the wearable terminal devices 12, patient information transmitted from the server device 15 by communication performed with the server device 15, and the like. The terminal information is information related to the wearable terminal device 12. The information related to the wearable terminal device 12 indicates, for example, information related to the state of progress of ophthalmic examination. The information related to the state of progress of ophthalmic examination includes the latest anterior segment image, state-of-progress information indicating the state of progress of visual field test, and eyewear worn/not-worn information indicating whether or not the patient is wearing the eyewear terminal device 16 correctly.

Processing transitions to step 416 when negative determination is made at step 402, i.e. when the device information has not been received. Processing transitions to step 404 when affirmative determination is made at step 402, i.e. when the device information has been received.

At step 404, the display control section 184 determines whether or not the received device information is the terminal information. Processing transitions to step 412 when negative determination is made at step 404, i.e. when the received device information is not the terminal information, namely, when the received device information is the patient information 154A. Processing transitions to step 406 when affirmative determination is made at step 404, i.e. when the received device information is terminal information.

At step 406, the display control section 184 determines whether or not information related to the received terminal information is being displayed on the state-of-progress screen 190. Processing transitions to step 408 when negative determination is made at step 406. i.e. when the information related to the received terminal information is not being displayed on the state-of-progress screen 190. Processing transitions to step 410 when affirmative determination is made at step 406, i.e. when the information relating to the received terminal information is being displayed on the state-of-progress screen 190.

At step 408, the display control section 184 causes the display 86A to start displaying the information related to the terminal information, and then processing transitions to step 416. The information related to the terminal information is thereby displayed on the state-of-progress screen 190.

As illustrated in the example of FIG. 13, the first state-of-progress screen 190A includes a terminal ID display region 190A1, a state-of-progress display region 190A2, an anterior segment image display region 190A3, an eyewear wearing state display region 190A4, and a patient information display region 190A5. Information related to the terminal information is displayed in the terminal ID display region 190A1, the state-of-progress display region 190A2, the anterior segment image display region 190A3, and the eyewear wearing state display region 190A4, and the patient information 164A is displayed in the patient information display region 190A5.

A terminal ID enabling unique identification of a first wearable terminal device 12 from out of the five wearable terminal devices 12 with established communication with the management device 14 is displayed in the terminal ID display region 190A1. In the present exemplary embodiment an eyewear ID of the eyewear terminal device 16 corresponding to the received terminal information is employed as the terminal ID.

The state of progress of visual field test is mainly displayed in the state-of-progress display region 190A2. In the example thereof illustrated in FIG. 13, information content of “VISUAL FIELD TEST SUBJECT: RIGHT EYE ONLY” is displayed as information enabling the visual field test subject eye to be identified, information content of “RIGHT EYE: BEING EXAMINED” is displayed as information enabling the examination subject eye undergoing the visual field test to be identified, and an indicator indicating the state of progress is displayed. The indicator is displayed in the state-of-progress display region 190A2 at a being examined position.

The patient's latest anterior segment image identified by the patient information 164 being displayed in the patient information display region 190A5 is displayed in the anterior segment image display region 190A3. The patient identified by the patient information 164A being displayed in the patient information display region 190A5 indicates, in other words, the patient who is currently using the wearable terminal device 12 identified by the terminal ID being displayed in the terminal ID display region 190A1. In the example illustrated in FIG. 13, the right-eye anterior segment image and the left-eye anterior segment image are displayed, with the anterior segment image of the left-eye that is not the examination subject eye displayed grayed out.

Information indicating whether or not the eyewear terminal device 16 is being worn by the patient is displayed in the eyewear wearing state display region 190A4. In the example illustrated in FIG. 13, information content of “BEING WORN” is displayed to indicate that the eyewear terminal device 16 is being worn by the patient. The background color of the eyewear wearing state display region 190A4 changes according to the state of progress. For example, the background color is a white, yellow, pink, or gray color. White indicates a state prior to the visual field test, yellow indicates a state during the visual field test, pink indicates that the visual field test has been completed, and gray indicates an examination subject eye has not yet been instructed for the visual field test.

In the example illustrated in FIG. 13, the first state-of-progress screen 190A is a screen corresponding to the wearable terminal device 12 including the eyewear terminal device 16 for which the terminal ID is “EA”. The second state-of-progress screen 190B is a screen corresponding to the wearable terminal device 12 including the eyewear terminal device 16 for which the terminal ID is “EC”. The third state-of-progress screen 190C is a screen corresponding to the wearable terminal device 12 including the eyewear terminal device 16 for which the terminal ID is “YV”. The fourth state-of-progress screen 190D is a screen corresponding to the wearable terminal device 12 including the eyewear terminal device 16 for which the terminal ID is “MI”. Furthermore, the fifth state-of-progress screen 190E is a screen corresponding to the wearable terminal device 12 including, the eyewear terminal device 16 for which the terminal ID is “GZ”.

The second state-of-progress screen 190B includes a terminal ID display region 190B1, a state-of-progress display region 190B2, an anterior segment image display region 190B3, an eyewear wearing state display region 190B4, and a patient information display region 190B5.

In the example illustrated in FIG. 13, a terminal ID enabling unique identification of a second wearable terminal device 12 from out of the five wearable terminal devices 12 with established communication with the management device 14 is displayed in the terminal ID display region 190B1. Information content of “EXAMINATION COMPLETED” is displayed in the state-of-progress display region 190B2. An indicator is displayed in the state-of-progress display region 190B2 at an examination completed position. The field-of-view defect map 240 is, as described above, displayed in the anterior segment image display region 190B3. Information content of “NOT BEING WORN” is displayed as information to indicate that the eyewear terminal device 16 is not being worn by a patient in the eyewear wearing, state display region 190B4.

The third state-of-progress screen 190C includes a terminal ID display region 190C1, a state-of-progress display region 190C2, an anterior segment image display region 190C3, an eyewear wearing state display region 190C4, and a patient information display region 190C5.

In the example illustrated in FIG. 13, a terminal ID enabling unique identification of a third wearable terminal device 12 from out of the five wearable terminal devices 12 with established communication with the management device 14 is displayed in the terminal ID display region 190C1. The information content of “RIGHT-EYE: BEING EXAMINED” is displayed in the state-of-progress display region 190C2. An indicator is displayed in the state-of-progress display region 190C2 at a being examined position. An anterior segment image of the patient identified by the patient information 164A being displayed on the patient information display region 190C5 is displayed in the anterior segment image display region 190C3. Information content of “NOT BEING WORN” and information content of “ERROR” are displayed as information to indicate that the eyewear terminal device 16 is not being worn by a patient in the eyewear wearing state display region 190C4. Note that displaying the information content of “ERROR” is implemented by execution of error processing at step 452, described later.

The fourth state-of-progress screen 190D includes a terminal ID display region 190D1, a state-of-progress display region 190D2, an anterior segment image display region 190D3, an eyewear wearing state display region 190D4, and a patient information display region 190D5.

In the example illustrated in FIG. 13, a terminal ID enabling unique identification of a fourth wearable terminal device 12 from out of the five wearable terminal devices 12 with established communication with the management device 14 is displayed in the terminal ID display region 190D1. Information content of “UNDER AUDIO GUIDANCE” is displayed in the state-of-progress display region 190D2. The “UNDER AUDIO GUIDANCE” indicates, for example, a state in which the patient is being guided by audio output from the speaker 140 by execution of the processing of step 258L illustrated in FIG. 9A or the processing of step 258S illustrated in FIG. 9B. The latest anterior segment image of the patient identified by the patient information 164A displayed in the patient information display region 190D5 is displayed in the anterior segment image display region 190D3. The information content of “BEING WORN” is displayed in the eyewear wearing state display region 190D4 as information to indicate that the eyewear terminal device 16 is being worn by the patient.

In the example illustrated in FIG. 13, the wearable terminal device 12 including the eyewear terminal device 16 with the terminal ID “GZ” is being charged, and so the information content “BEING CHARGED” is displayed in the fifth state-of-progress screen 190E as information to enable the status of being charged to be recognized visually Information content of “BATTERY 88%” and an indicator of the capacity of the battery is displayed in the fifth state-of-progress screen 190E as information indicating the capacity of the battery.

In the example illustrated in FIG. 13, due to there currently being only five devices connected in a communicable state with the management device 14 from out of the wearable terminal devices 12, the sixth state-of-progress screen 190F adopts a non-display state.

At step 410 illustrated in FIG. 11, the display control section 184 causes the display 86A to update the display content of information related to the terminal information, and the processing transitions to step 416. The display content of the terminal ID display region 190A1, the state-of-progress display region 190A2, the anterior segment image display region 190A3, and the eyewear wearing state display region 190A4 is thereby updated.

For example, when the eyewear terminal device 16 is taken off the patient, in the eyewear wearing state display region 190A4, “NOT BEING WORN” is displayed as the information content in the eyewear wearing state display region 190B4 of the second state-of-progress screen 190B. Furthermore, when the error processing of the step 452, described later, is executed, information content of “ERROR” is displayed so as to be indicated in the eyewear wearing state display region 190C4 of the third state-of-progress screen 190. Moreover, when the visual field test is complete, the information content of “EXAMINATION COMPLETED” is displayed so as to be indicated in the state-of-progress display region 190B2 of the second state-of-progress screen 190B, and a state is adopted in which the indicator is at the examination completed position. Moreover, when the visual field test has been completed, the information content of “EXAMINATION COMPLETED” is displayed so as to be indicated in the state-of-progress display region 190B2 of the second state-of-progress screen 190B, and a state is adopted in which the indicator is at an examination completed position. Furthermore, when under audio guidance from the speaker 140, the information content of “UNDER AUDIO GUIDANCE” is displayed so as to be indicated on the state-of-progress display region 190D2 of the fourth state-of-progress screen 190D.

At step 412, the display control section 184 determines whether or not the patient information 64A is in a non-display state. For example, the display control section 184 determines whether or not the patient information 64A related to the patient using the wearable terminal device 12 identified by the terminal ID being displayed in the terminal ID display region 190A1 is being displayed in the patient information display region 190A5.

Processing transitions to step 414 when affirmative determination is made at step 412, i.e. when the patient information 64A is in the non-display state. Processing transitions to step 416 when negative determination is made at step 412, i.e. when the patient information 64A is in the non-display state, namely when the patient information 64A is being displayed.

At step 414, the display control section 184 causes the display 86A to start displaying the patient information 64A, and then processing transitions to step 416. Thereby, for example, as long as there is patient information 64A related to the patient using the wearable terminal device 12 identified by the terminal ID being displayed in the terminal ID display region 190A1 then the patient information 64A is displayed in the patient information display region 190A5.

At step 416, the display control section 184 determines whether or not an end condition relating to display control processing has been satisfied. The end condition relating to display control processing indicates a condition to end the display control processing. Examples of the end condition relating to display control processing include a condition that a specific period of time has elapsed, a condition that the reception device 84 has received an end instruction, and/or a condition that a situation requiring the display control processing to be forcibly ended has been detected by the CPU 90.

Processing transitions to step 402 when negative determination is made at step 416, i.e. when the end condition relating to display control processing has not been satisfied. Processing transitions to step 418 when affirmative determination is made at step 416, i.e. when the end condition relating to display control processing has been satisfied.

At step 418, the display control section 184 causes the display 86A to end the display of the state-of-progress screen 190, and then ends the display control processing.

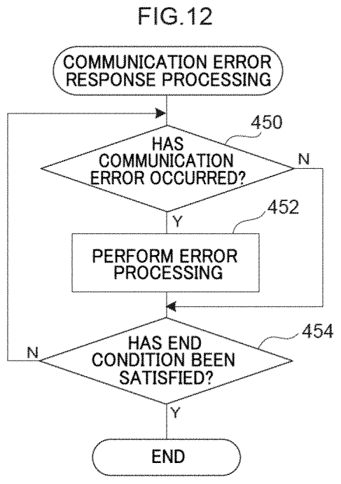

Next, explanation follows regarding communication error response processing implemented by the CPU 90 executing the communication error response program 94C by the start of execution of the terminal management processing, with reference to FIG. 12. In the following description of the communication error response processing, for ease of explanation, an example will be described of the wearable terminal device 12 identified by the terminal ID being displayed in the terminal ID display region 190C1 of the third state-of-progress screen 190C illustrated in FIG. 13, the management device 14, and the server device 15.

At step 450 in the communication error response processing illustrated in FIG. 12, the display control section 184 determines whether or not a communication error has occurred. The “communication error” referred to here indicates, for example, an error in the communication between the wearable terminal device 12 and the management device 14, or an error in the communication between the management device 14 and the server device 15. These errors in the communication indicate, for example, a phenomenon in which communication is interrupted at an unintentional timing.

Processing transitions to step 454 when negative determination is made at step 450, i.e, when a communication error is not occurring. Processing transitions to step 452 when affirmative determination is made at step 450, i.e. when a communication error has occurred.

At step 452, the display control section 184 executes error processing, and then processing transitions to step 454. The error processing indicates, for example, processing to control the display 86A so as to display information content of “ERROR” in the eyewear wearing state display region 190C4. Moreover, other examples of the error processing include processing to cause a speaker (not illustrated in the drawings) to output audio such as “A COMMUNICATION ERROR HAS OCCURRED”.

At step 454, the display control section 184 determines whether or not an end condition relating to communication error response processing has been satisfied. The end condition relating to communication error response processing indicates a condition to end the communication error response processing. Examples of the end condition relating to communication error response processing include a condition that a specific period of time has elapsed, a condition that the reception device 84 has received an end instruction, and/or a condition that a situation requiring the communication error response processing to be forcibly ended has been detected by the CPU 90.

Processing transitions to step 450 when negative determination is made at step 454, i.e. when the end condition relating to communication error response processing has not been satisfied. The communication error response processing is ended when affirmative determination is made at step 454, i.e. when the end condition relating to communication error response processing has been satisfied.

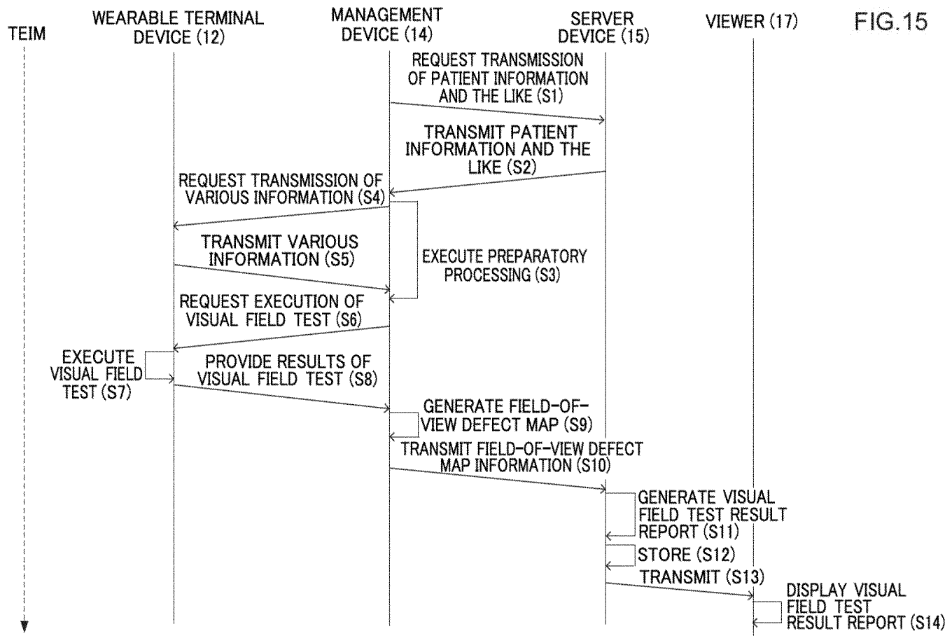

Next explanation follows regarding a flow of processing between the wearable terminal device 12, the management device 14, the server device 15, and the viewer 17, with reference to FIG. 15.

As illustrated in the example in FIG. 15, the management device 14 requests transmission of patient information and the like from the server device 15 (S1). The server device 15 transmits the patient information and the like to the management device 14 in response to the request from the management device 14 (S2).

On receipt of the patient information and the like transmitted from the server device 15, the management device 14 executes preparatory processing (S3). The preparatory processing referred to here indicates, for, example, the processing of step 212 to step 220 illustrated in FIG. 7A and FIG. 7B. In the preparatory processing, the management device 14 requests the wearable terminal device 12 to transmit various information (S4). The various information indicates, for example, information about the operational status of the wearable terminal device 12. The various information also indicates, for example, information as to whether or not imaging of the anterior segments of the subject eyes 44 has started, information as to whether or not the inter-pupil distance has been detected, and/or information as to whether or not the response button 19 has been pressed.

In response to the request from the management device 14, the wearable terminal device 12 transmits the various information to the management device 14 (S5). On completion of the preparatory processing, the management device 14 requests the wearable terminal device 12 to execute the visual field test (S6).

In response to the request from the management device 14, the wearable terminal device 12 executes the visual field test on the examination subject eye by executing the visual field test processing as illustrated in the example of FIG. 9A and FIG. 9B (S7). The wearable terminal device 12 provides visual field test results to the management device 14 (S8). The “VISUAL FIELD 1EST RESULTS” referred to here indicates, for example, mark projection position information and sensory information. Note that the “VISUAL FIELD TEST RESULTS” may be merely the mark projection position information related to the position of the mark projected at the timing when the response button 19 was pressed.

In the first exemplary embodiment, as illustrated in the example of FIG. 9B, the wearable terminal device 12 generates the field-of-view defect map information (see step 258V of FIG. 9B), however technology disclosed herein is not limited thereto. For example, as illustrated in FIG. 15, the management device 14 may generate the field-of-view defect map information.

Namely, in the example illustrated in FIG. 15, the management device 14 generates the field-of-view defect map 240 (see FIG. 1) based on the visual field test results (S9). Thus when the field-of-view defect map 240 is generated by the management device 14, the management device 14 transmits field-of-view defect map information that is information including the generated field-of-view defect map 240 to the server device 15 (S10).

The server device 15 receives the field-of-view defect map information transmitted from the management device 14, and then generates a visual field test result report indicating the results of the visual field test based on the field-of-view defect map information received (S11). Moreover, the server device 15 stores the generated visual field test result report in the secondary storage section 94 (S12). The server device 15 then transmits the generated visual field test result report to the viewer 17 (S13).

Note that a configuration may be adopted in which, not only is the field-of-view defect map 240 generated by the wearable terminal device 12 or the management device 14, but a field-of-view defect map is plotted in advance by the server device 15 so as to generate the visual field test result report. Moreover, for example, a configuration may be adopted in which, the field-of-view defect map is not generated only with the field-of-view defect map information for the same patient (patient having the same patient ID), but a field-of-view defect area is displayed in overlay on a fundus image, or a field-of-view defect area is displayed in overlay on a 3D-OCT image.

On receipt of the visual field test result report, the viewer 17 displays the received visual field test result report on the display 17C (S14).

Note that the processing by the viewer 17 at illustrated S14 is processing implemented by the CPU 17H reading the viewer-side program 17J1 and executing the read viewer-side program 17J1.

However, in conventional cases in which patients A to C visiting a hospital who have finished reception are treated in the sequence of visual field test→consultation→fundus imaging, the visual field test is performed in sequence for each of the patients on a one-by-one bases by a single medical service professional operating a single static visual field test device. Thus in the conventional example as illustrated in FIG. 24, treatment progresses sequentially for the patients A to C.

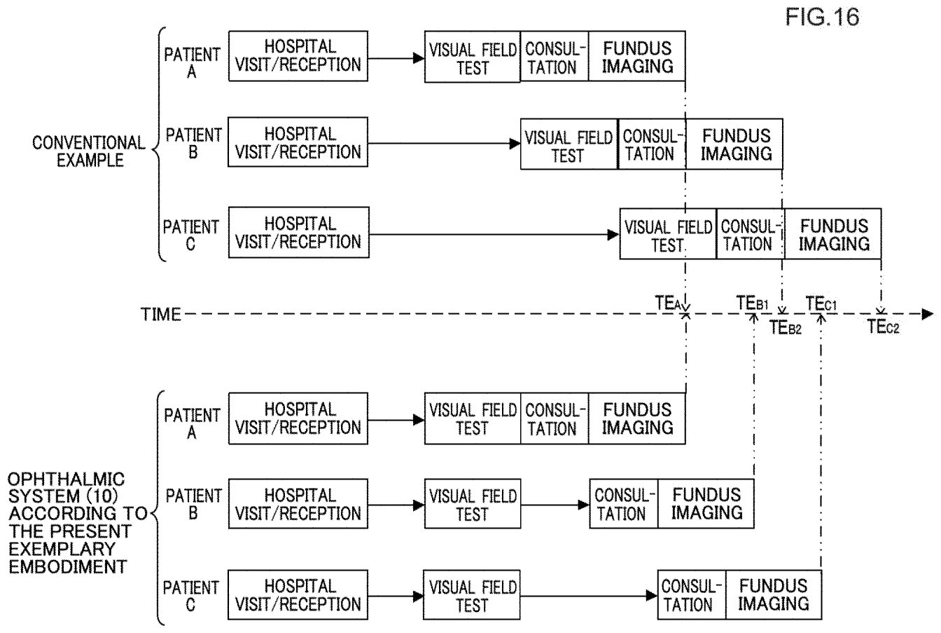

In contrast thereto, with the ophthalmic system 10 according to the present exemplary embodiment, the plural wearable terminal devices 12 are connected to the management device 14 so as to be capable of wireless communication therewith, enabling the management device 14 to perform unified management of the plural wearable terminal devices 12. As illustrated in the example of FIG. 16, this thereby enables the single medical service professional to carry out the visual field tests for the patients A to C in parallel.

As illustrated in the example of FIG. 16, when there are three patients visiting the hospital at the same time, the total time needed to perform treatment in the sequence visual field test→consultation→fundus imaging (hereafter referred to simply as “total time”) is reduced for the second person onwards. This is explained in more detail below.

If “TE_{A}” is the total time for the first patient when a conventional visual field test device is employed, then the total time for the first patient when the ophthalmic system 10 is employed is also “TE_{A}”. However, it “TE_{B2}” is the total time for the second patient when a conventional visual field test device is employed, then the total time for the second patient when the ophthalmic system 10 is employed is “TE_{B1}” (<TE_{B2}). Namely, when the ophthalmic system 10 is employed the second patient has a reduced time in the hospital of an amount “TE_{B2}−TE_{B1}” compared to the second patient when the conventional visual field test device is employed. Moreover, if “TE_{C2}” is the total time for the third patient when a conventional visual field test device is employed, then the total time for the third patient when the ophthalmic system 10 is employed is “TE_{C1}” (<TE_{C2}). Namely, when the ophthalmic system 10 is employed the third patient has a reduced time in the hospital of an amount “TE_{C2}−TE_{C1}” compared to the third patient when the conventional visual field test device is employed.

Moreover, employing the ophthalmic system 10 enables each patient to receive a consultation and ophthalmic imaging more quickly than in the conventional example. This not only reduces the burden on the patient, but is also advantageous on the ophthalmic side. This advantage is the advantage of enabling work to progress faster than hitherto. Thus from the ophthalmic side, employing the ophthalmic system 10 enables a chain of treatment of visual field test→consultation→fundus imaging to be performed for more patients than hitherto within the same consultation time as hitherto.

Moreover, due to the wearable terminal devices 12 being portable devices, they take up less installation space than a conventional static visual field test device. Furthermore, due to the wearable terminal devices 12 being portable devices, the visual field test can be performed in a waiting room or the like. Thus the wearable terminal devices 12 enable the time that a patient spends in a hospital to be reduced in comparison to cases in which a conventional static visual field test device is employed.

As explained above, the wearable terminal devices 12 are each equipped with the optical system 27 to guide the laser beams to the retina 46R and/or the retina 46L. The wearable terminal devices 12 are each also equipped with the control section 170 to control the optical system 27 such that the visual field test is performed on the retina 46R and/or the retina 46L by the laser beams being shone onto the retina 46R and/or the retina 46L. Thus the wearable terminal devices 12 are able to contribute to the efficiency of carrying out the visual field tests.

The wearable terminal devices 12 are each also equipped with the right-eye optical system 27R and the left-eye optical system 27L. Thus the wearable terminal devices 12 enable the visual field tests to be carried out on both eyes using the single laser light source 114.

The wearable terminal devices 12 are each also equipped with the scanner 28 to scan the laser beams, and the reflection mirror 42 to reflect the laser beams scanned by the scanner 28 onto the retinas 46. Thus even for patients with cataracts, namely, patients whose crystalline lenses are cloudy, the wearable terminal devices 12 enable the laser beams for visual field tests to be sensed visually.

Moreover, the wearable terminal devices 12 are each also equipped with the right-eye inward-facing camera 48R and the left-eye inward-facing camera 48L to image the anterior segments of the subject eyes 44. The control section 170 then detects the inter-pupil distance based on the right-eye anterior segment image and the left-eye anterior segment image obtained by imaging with the right-eye inward-facing camera 48R and the left-eye inward-facing camera 48L, and controls the position of the reflection mirror 42 based on the detected inter-pupil distance. The wearable terminal devices 12 thereby enable visual field tests to be carried out with good precision even though the inter-pupil distance varies between patients.

Moreover, the wearable terminal devices 12 are each also equipped with the response button 19 to receive operation to indicate whether or not the patient has sensed the laser beams when the laser beams have been shone onto the retinas 46. Moreover, the wearable terminal devices 12 are each also equipped with the output section 172 to output information in response to receipt of operation by the response button 19. In the first exemplary embodiment described above, the output section 172 transmits sensory information to the management device 14. The wearable terminal devices 12 thus thereby enable a medical service professional to easily ascertain positions on the retinas 46 that are not sensitive to the laser beams.

Moreover, the wearable terminal devices 12 are each also equipped with the wireless communication section 112 to perform communication with the management device 14 so as to enable the management device 14 to manage the visual field test. The wearable terminal devices 12 thereby enable a reduction to be achieved in the processing burden related to management of the visual field test.

Note that the management of the visual field tests is, for example, management including management of the laser beams used in the visual field tests, and including, by shining the laser beams onto the retinas 46, management of the sensory information to indicate that patients have visually sensed shone laser beams. The wearable terminal devices 12 thus thereby enable at least a reduction to be achieved in the processing related to managing the laser beams, employed in visual field tests and related to managing the sensory information.

The management device 14 is equipped with the providing section 180 to provide the examination subject eye instruction information and the patient information 64A to the wearable terminal device 12. The management device 14 is also equipped with the acquisition section 182 to acquire the sensory information from the wearable terminal device 12 by performing wireless communication with the wearable terminal devices 12. The management device 14 is thereby able to contribute to carrying out the visual field tests efficiently.

Moreover, the management device 14 is also equipped with the display control section 184 to control the display 86A so as to cause the state-of-progress screen 190 that accords with the state of progress of visual field test to be displayed on the display 86A. The wearable terminal devices 12 thereby enable a medical service professional to easily ascertain the state of progress of visual field tests.

Moreover, in the management device 14, the providing section 180 provides the examination subject eye instruction information and the patient information 64A to each of the wearable terminal devices 12 by performing wireless communication with each of the plural wearable terminal devices 12. The acquisition section 182 acquires the sensory information from each of the wearable terminal devices 12 by performing wireless communication with each of the plural wearable terminal devices 12. The wearable terminal devices 12 thereby enable a single medical service professional to carry out the visual field tests on plural patients in parallel.

### Second Exemplary Embodiment

Although in the first exemplary embodiment described above explanation has been given of a case in which a laser beam is shone from a single light source, in the second exemplary embodiment explanation will be given of a case in which laser beams are shone from each of two respective light sources.

Note that in the second exemplary embodiment the same reference numerals will be appended to configuration elements that are the same as those of the first exemplary embodiment and explanation thereof will be omitted, with explanation given of portions differing from the first exemplary embodiment.

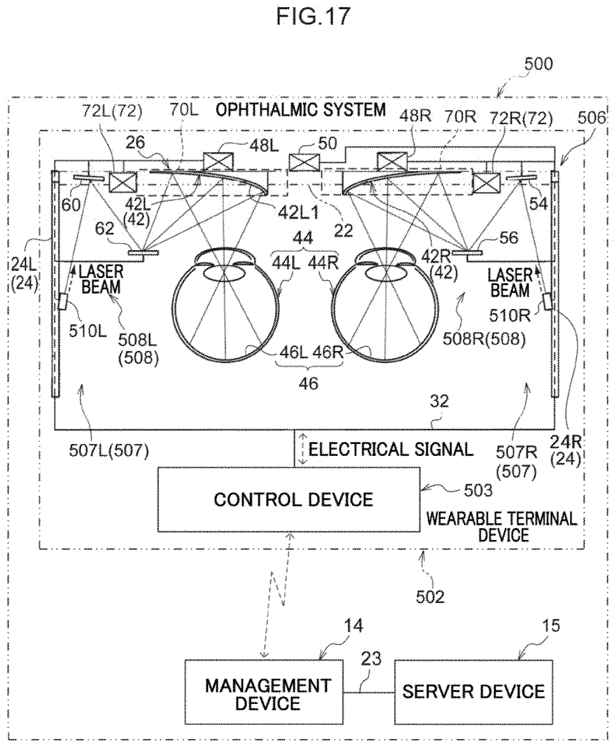

As illustrated in the example of FIG. 17, an ophthalmic system 500 according to the second exemplary embodiment differs from the ophthalmic system 10 in that it includes a wearable terminal device 502 instead of the wearable terminal device 12.

The wearable terminal device 502 differs from the wearable terminal device 12 in that it includes the control device 503 instead of the control device 18, it includes the eyewear terminal device 506 instead of the eyewear terminal device 16, and that it does not include the optical splitter 20. Moreover, the wearable terminal device 502 also differs from the wearable terminal device 12 in that it does not include the optical fibers 30, 38, 40. Note that, similarly to the ophthalmic system 10, the ophthalmic system 500 also includes plural of the wearable terminal devices 502, with each of the wearable terminal devices 502 being connected to the management device 14 so as to be capable of wireless communication therewith.

The eyewear terminal device 506 differs from the eyewear terminal device 16 in that it includes the optical system 507 instead of the optical system 27 and includes the scanner 508 instead of the scanner 28

The optical system 507 differs from the optical system 27 in that it includes the right-eye optical system 507R instead of the right-eye optical system 27R and in that it includes the left-eye optical system 507L instead of the left-eye optical system 27L. Moreover, the optical system 506 differs from the optical system 27 in that it includes the scanner 508 instead of the scanner 28.

The scanner 508 differs from the scanner 28 in that it includes the right-eye scanner 508R instead of the right-eye scanner 28R, and includes the left-eye scanner 508L instead of the left-eye seamier 28L.

The right-eye scanner 508R differs from the right-eye scanner 28R in that it includes a right-eye laser light source 510R instead of the right-eye illumination section 52. The right-eye laser light source 510R is an example of a right-eye laser light source according to technology disclosed herein. The right-eye laser light source 510R emits a laser beam towards the MEMS mirror 54 similarly to the right-eye illumination section 52. The right-eye laser light source 510R is connected to the bus line 32 through a right-eye laser light source control circuit (not illustrated in the drawings) and operates under control from the CPU 120. The right-eye laser light source control circuit is a driver to control the right-eye laser light source 510R according to the instructions of the CPU 120.

The left-eye scanner 508L differs from the left-eye scanner 28L in that it includes a left-eye laser light source 510L instead of the left-eye illumination section 58. The left-eye laser light source 510L is an example of a left-eye light source according to technology disclosed herein. The left-eye laser light source 510L emits a laser beam towards the MEMS mirror 60 similarly to the left-eye illumination section 58. The left-eye laser light source 510L is connected to the bus line 32 through a left-eye laser light source control circuit (not illustrated in the drawings) and operates under control from the CPU 120. The right-eye laser light source control circuit is a driver to control the left-eye laser light source 510L according to the instructions of the CPU 120.

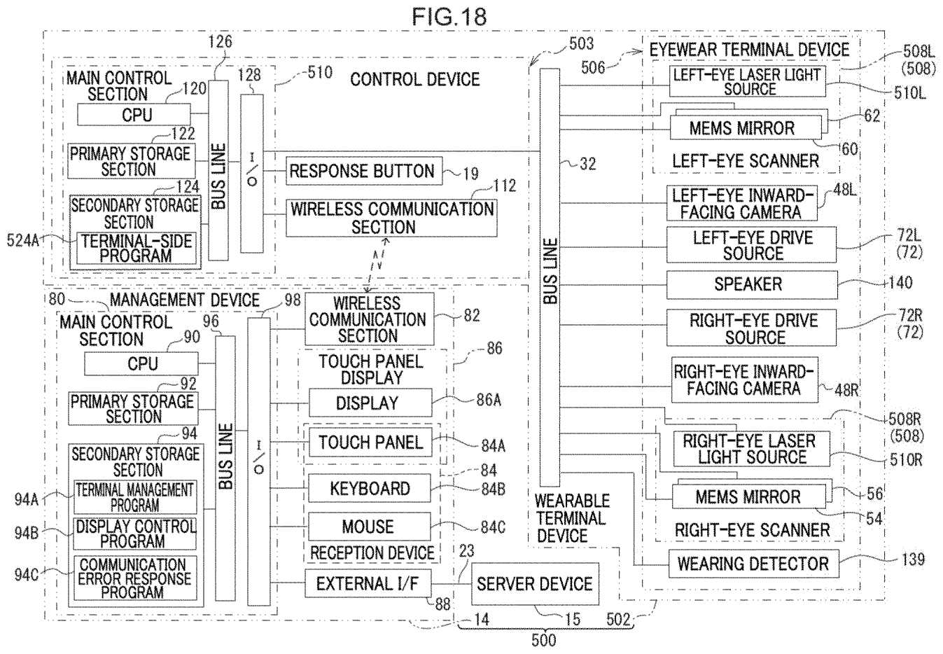

As illustrated in the example of FIG. 18, the control device 503 differs from the control device 18 in that it includes a main control section 510 instead of the main control section 110. The main control section 510 differs from the main control section 110 in that it stores a terminal-side program 524A in the secondary storage section 124 instead of the terminal-side program 124A.

The CPU 120 reads the terminal-side program 524A from the secondary storage section 124, and expands the read terminal-side program 524A into the primary storage section 162. The CPU 120 executes the terminal-side program 524A target has been expanded into the primary storage section 122.

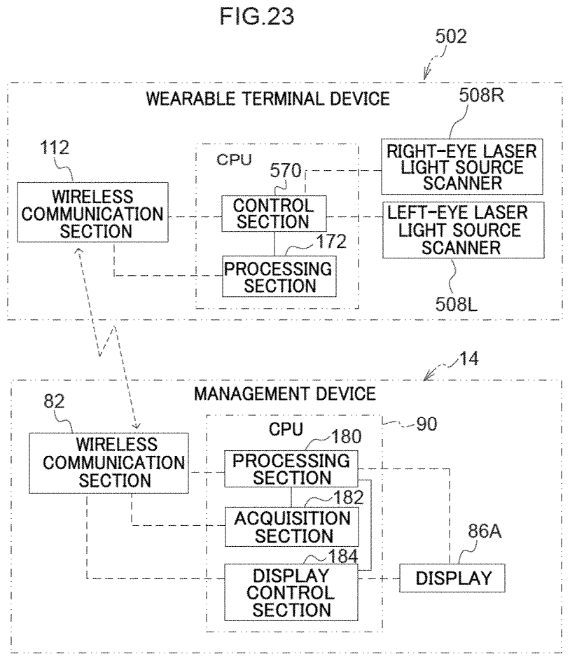

As illustrated in the example of FIG. 23, the CPU 120 operates as a control section 570 and the output section 172 by executing the terminal-side program 524A.

The control section 570 controls the right-eye laser light source 510R and the left-eye laser light source 510L such that the visual field tests are performed on the retina 46R and/or the retina 46L by the right-eye laser beam and/or the left-eye laser beam being supplied into the optical system 507. The right-eye laser beam is an example of right-eye light according to technology disclosed herein, and the left-eye laser beam is an example of left-eye light according to technology disclosed herein. Note that the right-eye laser beam indicates a laser beam from the right-eye laser light source 510R. The left-eye laser beam indicates a laser beam from the left-eye laser light source 510L.

Note that in the wearable terminal device 502 according to the second exemplary embodiment, the right-eye laser light source 510R is usable when a right-eye laser light source flag is switched ON, and the left-eye laser light source 510L is usable when a left-eye laser light source flag is switched ON. For ease of explanation, when there is no need to discriminate in the description between the right-eye laser light source flag and the left-eye laser light source flag they will be referred to as “laser light source flags”.

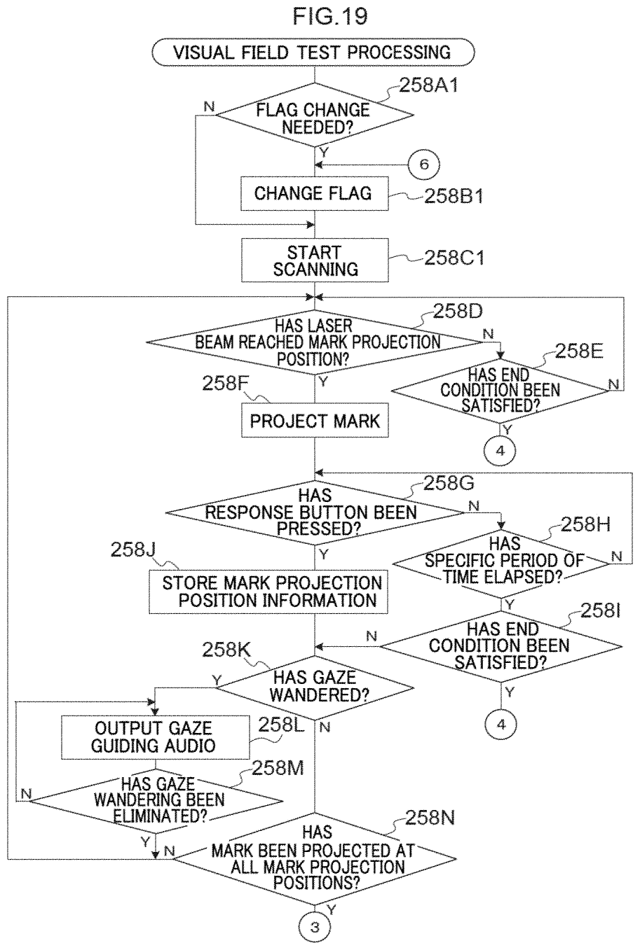

Explanation next follows regarding terminal-side processing implement by the CPU 120 executing the terminal-side program 524A when the main power source (not illustrated in the drawings) of the wearable terminal device 502 has been turned on, with reference to FIG. 19 and FIG. 9B.

Note that, for ease of explanation, processing the same as that of the terminal management processing according to the first exemplary embodiment will be appended with the same step number, and explanation thereof will be omitted.

Note that the terminal-side processing according to the second exemplary embodiment differs from the terminal-side processing according to the first exemplary embodiment in that it includes a step 258A1 instead of the step 258A, and includes a step 258B1 instead of the step 258B. Moreover, the terminal-side processing according to the second exemplary embodiment differs from the terminal-side processing according to the first exemplary embodiment in that it includes a step 258C1 instead of the step 258C, and includes a step 258U1 (see FIG. 9B) instead of the step 258U.

At step 258A1 illustrated in FIG. 19, the control section 570 determines whether or not a currently ON laser light source flag needs to be changed based on the examination subject eye instruction information in the previously mentioned required information included in the visual field test instruction information.

Processing transitions to step 258C1 when negative determination is made at step 258A1, i.e. when there is no need to change the currently ON laser light source flag. Processing transitions to step 258B1 when affirmative determination is made at step 258A1, i.e. when the currently ON laser light source flag needs to be changed.

At step 258A1, the control section 570 changes the laser light source flag based on the examination subject eye instruction information in the previously mentioned required information included in the visual field test instruction information, and then processing transitions to step 308. The “changing of the laser light source flag” referred to here indicates switching a laser light source flag that is ON to OFF, or changing a laser light source flag that is OFF to ON.

For example, the right-eye laser light source flag is ON and the left-eye laser light source flag is OFF when scanning is being performed on the retina 46R with a laser beam. Moreover, the left-eye laser light source flag is ON and the right-eye laser light source flag is OFF when scanning is being performed on the retina 46L with a laser beam.

At step 258C1, the control section 570 causes the laser beam to start being shone from the laser light source corresponding to the laser light source flag currently in an ON state from out of the right-eye laser light source 510R and the left-eye laser light source 510L, so as to start scanning the laser beam onto the retina 46. For example, when the right-eye laser light source flag is currently ON, scanning of the retina 46R with the right-eye laser beam is started by starting to shine the right-eye laser beam from the right-eye laser light source 510R. Moreover, for example, when the left-eye laser light source flag is currently ON, then scanning of the left-eye laser beam onto the retina 46L is started by starting to shine the left-eye laser beam from the left-eye laser light source 510L.

At step 258U1 illustrated in FIG. 9B, the control section 570 controls the right-eye laser light source 510R when the retina 46R is being scanned by the right-eye laser beam so as to end scanning by the right-eye laser light source 510R. The control section 570 also controls the left-eye laser light source 510L when the retina 46L is being scanned by the left-eye laser beam so as to end scanning by the left-eye laser light source 510L.

As described above, the wearable terminal device 502 is equipped with the optical system 507 to guide the right-eye laser beam to the retina 46R and to guide the left-eye laser beam to the retina 46L. The wearable terminal device 502 is equipped with the control section 570 to control the right-eye laser light source 510R and the left-eye laser light source 510L so as to perform visual field tests on the retina 46R and/or the retina 46L by supplying the right-eye laser beam and/or the left-eye laser beam into the optical system 507. The wearable terminal device 502 is thereby able to contribute to carrying out the visual field tests efficiently.

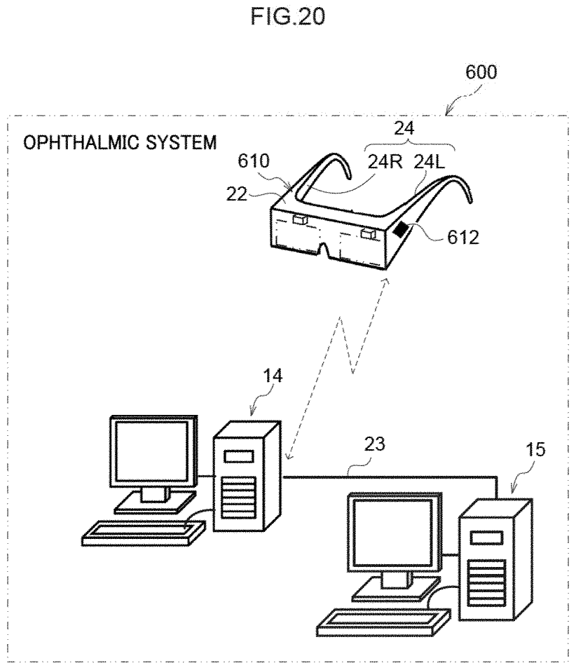

Note that although an example is given in the first exemplary embodiment of the wearable terminal device 12 in which the control device 18 and the optical splitter 20 are external to the eyewear terminal device 16, technology disclosed herein is not limited thereto. For example, an ophthalmic system 600 as illustrated in FIG. 20 may be employed instead of the ophthalmic system 10.

The ophthalmic system 600 differs from the ophthalmic system 10 in that it does not include the control device 18, the optical splitter 20, nor the cables 25, 34, 36. The ophthalmic system 600 also differs from the ophthalmic system 10 in that it includes an eyewear terminal device 610 instead of the eyewear terminal device 16.

The eyewear terminal device 610 includes a controller 352 that is a device with functionality equivalent to that of the control device 18 and a device with functionality equivalent to that of the optical splitter 20 integrated together and housed in the left temple piece 24L. In such a configuration cables equivalent to the cables 34, 36 are also housed in the frame of the eyewear terminal device 350. The frame of the eyewear terminal device 350 indicates, for example, the rim piece 22 and the temple piece 24.

An example of a method to detect an answer-response with the eyewear terminal device 610 is a method in which an answer-response is detected by a touch sensor (not illustrated in the drawings) provided in the temple piece 24 being touched by a patient. Another example of a method to detect an answer-response with the eyewear terminal device 610 is a method in which an answer-response is detected using a voice recognition device. In such cases, for example, the voice recognition device detects an answer-response by recognizing the “YES” (an expression of decision that a mark (light) has been sensed) or the “NO” (an expression of decision that a mark (light) has not been sensed) of a patient. Moreover, a configuration may be adopted in which the patient is required to grip a separately configured response button 19, such that a response result of the response button 19 is transmitted to the eyewear terminal device 610.

The controller 352 may be provided in the right temple piece 24R. Moreover, a configuration may be adopted in which a device with functionality equivalent to that of the control device 18 and a device with functionality equivalent to that of the optical splitter 20 are separately housed in the frame of the eyewear terminal device 350. In such cases, cable equivalent to that of the cable 25, namely, the cable connecting together the device with functionality equivalent to that of the control device 18 and the device with functionality equivalent to that of the optical splitter 20, is also housed in the frame of the eyewear terminal device 350.

The eyewear terminal device 610 thereby renders the cables 25, 34, 36 and the optical splitter 20 redundant, enabling a contribution to be made to greater compactness of the device overall.

Note that the wearable terminal device 500 according to the second exemplary embodiment is also configurable as a wireless wearable terminal device as in the wearable terminal device 610 illustrated in FIG. 20. Namely, a configuration may be adopted in which the wearable terminal device incorporates an eyewear terminal device including at least the optical system 507 from out of the devices equivalent to the right-eye laser light source 510R, the left-eye laser light source 510L, the optical system 507, and the control device 503. Such a configuration also enables a contribution to be made to greater compactness of the device overall.

Moreover, although the shutter 121 has been given as an example in the first exemplary embodiment, the technology disclosed herein is not limited thereto, and, instead of the shutter 121, a device may be employed that is capable of being controlled so as to let light pass through, such as a liquid crystal shutter.

Moreover, although laser beam have been given as examples in each of the exemplary embodiments described above. technology disclosed herein is not limited thereto, and, for example, light from super luminescent diodes may be employed instead of laser beams.

Moreover, although the response button 19 has been given as an example in each of the exemplary embodiments described above, the technology disclosed herein is not limited thereto. For example, instead of the response button 19, a touch panel display, keyboard, or a mouse or the like may be employed.

Moreover, although examples have been given in the exemplary embodiments described above in which the field-of-view defect map is generated by the wearable terminal device 12 (502), the technology disclosed herein is not limited thereto. For example, as illustrated in FIG. 15, the field-of-view defect map may be generated by the management device 14. In such cases, for example, a configuration may be adopted in which the processing section 171 generates correspondence information corresponding sensory information with mark projection position information related to the sensory information, transmits the generated correspondence information to the management device 14 through the wireless communication section 112, and the management device 14 generates a field-of-view defect map based on the correspondence information. Note that the mark projection position information related to the sensory information indicates mark projection position information corresponding to the position where the mark was being projected at the timing the response button 19 was pressed. Alternatively, a configuration may be adopted in which the processing section 171 transmits the mark projection position information corresponding to the position where the mark was being projected at the timing the response button 19 was pressed to the management device 14 through the wireless communication section 112, and the management device 14 generates the field-of-view defect map information based on the mark projection position information.

Moreover, although examples have been given in which the MEMS mirrors 54, 56, 60, 62 were employed in the exemplary embodiment described above, the technology disclosed herein is not limited thereto. For example, instead of the MEMS mirrors 54, 56, 60, 62, or together with one or more of the MEMS mirrors 54, 56, 60, 62, a mirror such as a galvanometer mirror and/or a polygon mirror or the like that enables electrical control of the position on the reflection face may be employed.

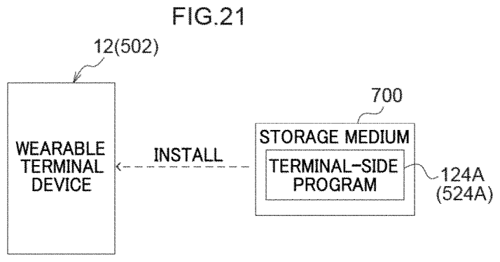

Moreover, although examples have been given in the exemplary embodiments described above in which the terminal-side program 124A (524A) is read from the secondary storage section 124, the terminal-side program 124-A (524A) does not necessarily have to be initially stored on the secondary storage section 124. For example, as illustrated in FIG. 21, a configuration may be adopted in which the terminal-side program 124A (524A) is first stored on a freely selected portable storage medium 700 such as an SSD, USB memory, or DVD-ROM or the like. In such a configuration the terminal-side program 124A (524A) on the storage medium 700 is then installed on the wearable terminal device 12 (502), and the installed terminal-side program 124A (524A) then executed by the CPU 120.

Moreover, a configuration may be adopted in which the terminal-side program 124A (524A) is stored on a storage section of another computer or server device or the like connected to the wearable terminal device 12 (502) over a communication network (not illustrated in the drawings), such that the terminal-side program 124A (524A) is then installed in response to a request from the wearable terminal device 12 (502). In such a configuration, the installed terminal-side program 124A (524A) is then executed by the CPU 120.

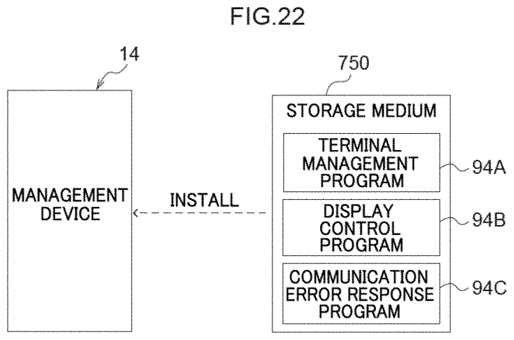

Moreover, although explanation has been given in the exemplary embodiment described above in which the management device-side program is read from the secondary storage section 94, the management device-side program does not necessarily have to be initially stored on the secondary storage section 94. For example, a configuration may be adopted in which, as illustrated in FIG. 22, the management device-side program is first stored on a freely selected portable storage medium 750 such as an SSD, USB memory, or DVD-ROM or the like. In such a configuration the management device-side program on the storage medium 750 is then installed on the management device 14, and the installed management device-side program is then executed by the CPU 90.

Moreover, a configuration may be adopted in which the management device-side program is stored on a storage section of another computer or server device or the like connected to the management device 14 over a communication network (not illustrated in the drawings), such that the management device-side program is then installed in response to a request from the management device 14. In such a configuration, the installed management device-side program is then executed by the CPU 90.

Moreover, the terminal management processing, the terminal-side processing, the server-side processing, the display control processing, and the communication error response processing in the exemplary embodiment described above are merely given as examples thereof. Thus obviously steps that are not required may be removed, new steps may be added, and the sequence of processing may be switched around within a range not departing from the spirit thereof.

Moreover, although examples are given in the exemplary embodiments described above of cases in which the terminal management processing, the terminal-side processing, the server-side processing, the display control processing, and the communication error response processing are implemented by a software configuration utilizing a computer, the technology disclosed herein is not limited thereto. For example, instead of a software configuration utilizing a computer, one or more processing of the terminal management processing, the terminal-side processing, the server-side processing, the display control processing, and the communication error response processing may be executed by a purely hardware configuration, i.e. a FPGA, ASIC, configuration or the like. One or more type of processing from out of the terminal management processing, the terminal-side processing, the server-side processing, the display control processing, and the communication error response processing may also be executed by configuration combining a software configuration and a hardware configuration.

Namely, examples of hardware resources to execute the various types of processing such as the terminal management processing, the terminal-side processing, the server-side processing, the display control processing, and the communication error response processing include CPUs that are general purpose processors that function as hardware resources to execute various types of processing by executing programs. Moreover, other examples of hardware resources include dedicated electronic circuits that are processors including circuit configurations such as FPGA, PLD, and ASIC configurations of dedicated design. Moreover, electronic circuits that combine circuit elements such as semiconductor elements and the like may also be employed as hardware structures of such processors. The hardware resources to execute the various types of processing may be one type from out of the plural types of processor described above, or a combination may be adopted of two or more processors that are of the same type or of a different type.

Moreover, for the processing section 180, the acquisition section 182, and the display control section 184 of the management device 14 in the example illustrated in FIG. 14, application may be made to a management device that, instead of being connected to wearable ophthalmic instruments, is connected in a communicable manner to a device including visual field test functionality capable of observing both eyes in a static device (for example, a static ophthalmic instrument). Namely, the processing executed by the management device 14 is also executable on a static device including visual field test functionality capable of observing both eyes.

In the present specification, “A and/or B” has the same meaning as “at least one out of A or B”. Namely, “A and/or B” may mean only A, may mean only B, or may mean a combination of A and B. Moreover, in the present specification, an expression in which three or more terms are linked together with “and/or” should be interpreted in a similar manner to “A and/or B”.

All publications, patent applications and technical standards mentioned in the present specification are incorporated by reference in the present specification to the same extent as if each individual publication, patent application, or technical standard was specifically and individually indicated to be incorporated by reference.

1. An ophthalmic instrument comprising:
a control device including a light source and a control section; and
an eyewear terminal equipped with an optical system that includes a right-eye optical system to guide light from the light source onto a right-eye retina and a left-eye optical system to guide light from the light source onto a left-eye retina;
the eyewear terminal and the control device being connected together by a cable including an optical fiber to supply light from the light source to the eyewear terminal; and
the control section executing a visual field test by controlling the optical system based on mark projection position information of a plurality of marks for the visual field test.

2. The ophthalmic instrument of claim 1, wherein the control section controls the light source and the optical system based on the mark projection position information so that visual field test light is projected on a retina of an examination subject eye for the visual field test.

3. An ophthalmic instrument comprising:
a control device including a control section; and
an eyewear terminal equipped with an optical system to guide right-eye light that is light from a right-eye light source onto a right-eye retina of a subject and to guide left-eye light that is light from a left-eye light source onto a left-eye retina of the subject;
the eyewear terminal and the control device being connected together by a cable including an optical fiber to supply the right-eye light from the right-eye light source and the left-eye light from the left-eye light source to the eyewear terminal; and
the control section transmitting a control signal, to control the optical system based on the mark projection position information of a plurality of marks for a visual field test, to the eyewear terminal with the cable and executing the visual field test.

4. An ophthalmic instrument comprising:
an eyewear terminal including:
a right-eye light source,
a left-eye light source,
an optical system to guide right-eye light that is light from the right-eye light source onto a right-eye retina of a subject and to guide left-eye light that is light from the left-eye light source onto a left-eye retina of the subject, and
a control section to control the right-eye light source, the left-eye light source, and the optical system based on mark projection position information of a plurality of marks for a visual field test.

5. The ophthalmic instrument of claim 1, wherein the optical system includes:
a right-eye scanner to scan visual field test light that is light arising from a light source employed in visual field test onto the right eye retina; and
a left-eye scanner to scan visual field test light that is light arising from a light source employed in visual field test onto the left eye retina.

6. The ophthalmic instrument of claim 5, wherein:
the ophthalmic instrument further comprises an anterior segment camera to image an anterior segment of a subject eye;
the right-eye scanner includes a right-eye reflection member to guide the visual field test light onto the right eye retina;
the left-eye scanner includes a left-eye reflection member to guide the visual field test light onto the left eye retina; and
the control section detects an inter-pupil distance based on an anterior segment image obtained by imaging with the anterior segment camera, and controls a position of the right-eye reflection member and/or the left-eye reflection member based on the detected inter-pupil distance.

7. The ophthalmic instrument of claim 6, wherein the anterior segment camera includes a right-eye camera to image an anterior segment of a right eye and a left-eye camera to image an anterior segment of a left eye.

8. The ophthalmic instrument of claim 1, wherein:
the ophthalmic instrument further comprises a response section connected to the control section; and
the control section combines response information received by the response section with the mark projection position information, and transmits the combination to an external device.

9. A management device comprising:
a communication section to exchange data with an ophthalmic instrument;
a processing section to generate transmission data for transmitting to the ophthalmic instrument by the communication section and to process received data received by the communication section; and
an acquisition section to acquire examination result information representing results of a visual field test employing the ophthalmic instrument;
the ophthalmic instrument including a light source, an optical system, a control section, and a response section;
the optical system including a right-eye optical system to guide light from the light source onto a right-eye retina of a subject and a left-eye optical system to guide light from the light source onto a left-eye retina of the subject;
the control section controlling the optical system;
the response section receiving operation by a user of the ophthalmic instrument when the user responds to having sensed light from the light source;
the transmission data including at least instruction information to instruct which is an examination subject eye for the visual field test from out of two eyes of the subject;
the received data including at least state-of-progress information about a state of progress of the visual field test and a response signal of the response section.

10. The management device of claim 9, further comprising a display control section to generate a state-of-progress screen according to the state of progress of the visual field test, and to output an image signal expressing an image including the generated state-of-progress screen.

11. The management device of claim 10, further comprising a display section to display the state-of-progress screen based on the image signal.

12. The management device of claim 9, wherein:
the ophthalmic instrument is a wearable ophthalmic instrument;
the management device includes are a plurality of wearable ophthalmic instruments;
the communication section transmits the instruction information to each of the wearable ophthalmic instruments by communicating with each of the wearable ophthalmic instruments; and
the acquisition section acquires the examination result information from each of the wearable ophthalmic instruments by communicating with each of the ophthalmic instruments through the communication section.

13. A method of managing an ophthalmic instrument, the ophthalmic instrument management method comprising:
a step of transmitting instruction information to instruct which is an examination subject eye from out of two eyes of a subject for a visual field test employing the ophthalmic instrument; and
a step of acquiring examination result information representing results of the visual field test;
the ophthalmic instrument including:
a control device that includes a light source, a response section, and a control section, and
an eyewear terminal equipped with an optical system including a right-eye optical system to guide a light from the light source onto a right-eye retina and a left-eye optical system to guide the light from the light source onto a left-eye retina;
the eyewear terminal and the control device being connected together by a cable including an optical fiber to supply light from the light source to the eyewear terminal;
the control section controlling the optical system; and
the response section receiving operation by a user of the ophthalmic instrument when the user responds to having sensed light from the light source.
